SEQUENCE LISTING (12 sequences)

SEQ ID NO: 1 - DNA, 39 bp, Artificial Sequence
  Synthetic
ctgtatgttctttgttctggtagatagcttgtacttggg

SEQ ID NO: 2 - DNA, 17 bp, Artificial
  Synthetic
ttccagtcttgctaacc

SEQ ID NO: 3 - DNA, 41 bp, Artificial sequence
  Synthetic
tgcagttggtattgatcttggtagcttggagtggttcggtt

SEQ ID NO: 4 - DNA, 16 bp, Artificial sequence
  Synthetic
ctcttgtgtgggtgtt

SEQ ID NO: 5 - DNA, 39 bp, Artificial sequence
  Synthetic
gggacaatgcacaagttataatcaaacccaagctgctct

SEQ ID NO: 6 - DNA, 16 bp, Artificial sequence
  Synthetic
tggaggaagagagggt

SEQ ID NO: 7 - DNA, 38 bp, Artificial sequence
  Synthetic
cagcagggcagaataaaaacgagaagaacagatggaaa

SEQ ID NO: 8 - DNA, 18 bp, Artificial sequence
  Synthetic
gggaggcaaagaaaggaa

SEQ ID NO: 9 - DNA, 36 bp, Artificial sequence
  Synthetic
gtggtgaaaggagaggtattggtggtccagtcgatt

SEQ ID NO: 10 - DNA, 17 bp, Artificial sequence
  Synthetic
cacagcaacaagggaag

SEQ ID NO: 11 - DNA, 38 bp, Artificial sequence
  Synthetic
ttgactgaacagagtgtgggattggggaaatggtctgg

SEQ ID NO: 12 - DNA, 17 bp, Artificial sequence
  Synthetic
tgaaatgaaggctgtgg

(19) United States
(12) Patent Application Publication
(10) Pub. No.: US 2007/0281299 A1
(43) Pub. Date: Dec. 6, 2007

(54) Diagnostic Test For Parkinson's Disease

(76) Inventors: Moussa B.H. Youdim, Haifa (IL); Silvia A. Mandel, Haifa (IL); Edna Grunblatt, Marktheidenfeld (DE); Peter Riederer, Wurzburg (DE)

Correspondence Address: BROWDY AND NEIMARK, P.L.L.C.;624 NINTH STREET, NW, SUITE 300, WASHINGTON, DC, 20001-5303, US

(21) Appl. No.: 10/586,523

(22) PCT Filed:
(86) PCT No.: PCT/IL05/00064

(30) Foreign Application Priority Data
Jan. 19, 2004 (EP) 04000968.0
Aug. 6, 2004 (EP) 04018771.8

(51) Int. Cl. C12Q 1/68; G01N 33/49; G01N 33/563

(52) U.S. Cl. 435/6; 436/512; 436/94

(57) ABSTRACT

The present invention relates to molecular markers for detection, prognosis and follow up of Parkinson's disease (PD), wherein said molecular markers are one or more genes with altered expression pattern, or gene products thereof (RNA or protein). Genes which expression is upregulated or downregulated in PD patients are tools for early diagnosis of PD, for monitoring the progress of the disease and can serve as targets for screening new agents for treatment of PD.

### FIELD OF THE INVENTION

The present invention relates to the use of molecular markers for detection, prognosis and follow up of Parkinson's disease.

Abbreviations: ACTB: beta actin; AD: Alzheimer's disease; ALAS1: aminolevulinate delta synthase 1; ALDH1A1: aldehyde dehydrogenase 1 family, member A1; ALS: Amyotrophic Lateral Sclerosis; ARPP: 21-cyclic AMP-regulated phosphoprotein; DA: dopamine; DEPC: diethyl pyrocarbonate; EGLN1: egl nine homolog 1; EIF4G1: eukaryotic translation initiation factor 4 gamma, 1; GAPDH: glyceraldehyde-3-phosphate dehydrogenase; HSPA8/HSC70/HSC54: chaperone heat shock 70 kDa protein 8; L13a: RPL13A; LGALS9: lectin, galactoside binding, soluble, 9; LOC56920: semaphorin sem2; LRP6: low density lipoprotein receptor-related protein 6; MAN2B1: mannosidase, alpha, class 2B, member1; MPTP: N-methyl-4-phenyl-1,2,3,6-tetrahydropyridine; OS: Oxidative stress; PARVA: parvin, alpha; pc: pars compacta; PD: Parkinson's disease; PENK: proenkephalin; PET: positron emission tomography; PMD: post-mortem delay; ROS: Reactive oxygen species; SELPLG: selectin P ligand; SKP1A: S-phase kinaseassociated protein 1A; SN: Substantia nigra; SPECT: single photon emission tomography; SPHK1: sphingosine kinase 1; SRPK2: SFRS protein kinase 2; SRRM2: serine/arginine repetitive matrix 2; TMEFF1: transmembrane protein with EGF-like and two follistatin-like domains 1; TRIM36: tripartite motif-containing 36; UCHL: 1-ubiquitin C-terminal hydrolase-L1; UPS-ubiquitin-proteasome system; UPDRS: Unified Parkinson's Disease Rating Scale; VMAT: vesicular monoamine member; ZSIG11: putative secreted protein ZSIG11.

### BACKGROUND OF THE INVENTION

Parkinson's disease (PD) is a progressive neurodegenerative disorder, with a prevalence of 1% in the population above 65 years of age, that results in degeneration of dopamine neurons in the substantia nigra (SN), and a consequent striatal dopamine deficiency (Bernheimer et al., 1973). The causes and mechanism for the degeneration of dopaminergic neurons is still elusive. Sporadic (non-hereditary) Parkinson's disease constitutes the most common form of the disease.

Both hereditary and sporadic forms of PD primarily converge in impairment of protein handling, catabolism and in oxidative-stress damage.

Potential contributing factors to the etiology of PD include ongoing selective oxidative stress (OS) resulting from mitochondrial dysfunction, auto-oxidation or enzymatic (monoamine-oxidase) oxidation of dopamine (DA) and excessive iron accumulation in the SN pars compacta (pc) (Riederer et al., 1989; Youdim et al., 1993; Gotz et al., 1994; Jenner and Olanow, 1996; Olanow and Youdim, 1996; Youdim and Riederer, 1997; Jenner, 1998). Specifically, redox-active iron has been observed within the melanin containing neurons that selectively die and in the rim of Lewy body, the morphological hallmark of PD.

Lewy body is composed of lipids, aggregated alpha synuclein (concentrating in its peripheral halo) and ubiquitinated, hyperphosphorylated neurofilament proteins (Jellinger, 2003). A number of studies (Ostrerova-Golts et al., 2000; Ebadi et al., 2001; Turnbull et al., 2001) have shown that alpha synuclein forms toxic aggregates in the presence of iron and this is considered to contribute to the formation of Lewy body via OS. Recently, Lewy bodies with aggregated ubiquitinated proteins are beginning to be viewed as a defensive measure aimed at removing toxic misfolded damaged proteins, rather than a mere general site for precipitated misfolded proteins (Hashimoto et al., 2004; Tanaka et al., 2004).

Misregulation of brain iron metabolism has taken center stage in neurodegenerative diseases since a significant number of mutated iron metabolism genes have now been shown to be directly involved in neurodegeneration (Felletschin et al., 2003; Youdim and Riederer, 2004). Thus, iron redox status constitutes a pivotal factor contributing to the extent of protein misfolding and aggregation in the ageing and disease affected brain.

Defects in ubiquitination and proteasomal protein handling are common features in PD and other chronic neurodegenerative diseases such as Alzheimer's disease (AD), Amyotrophic Lateral Sclerosis (ALS) and Huntington disease, and in ageing (Ciechanover and Brundin, 2003; Dawson and Dawson, 2003). This may, in turn, lead to impairment in several cellular processes linked to ubiquitination such as cell cycle, processing and regulation of transcription, intracellular trafficking, signalling pathways and degradation of normal and damaged intracellular proteins (Ciechanover and Brundin, 2003).

Accumulating evidence points out to a crucial role for protein misfolding and aggregation into protein inclusions in sporadic PD, which constitutes the most common form of the disease. For example, a loss of 20S proteasome alpha-subunits (McNaught et al., 2002; McNaught et al., 2003) and reduction in the activity of the 26/20S proteasome system in SNpc of sporadic PD (McNaught et al., 2003) has also been reported.

Three apparently independent gene mutations in alpha synuclein, parkin and ubiquitin C-terminal hydrolase-L1 (UCHL-1), which are capable of impairing the activity of the ubiquitin-proteasome system (UPS), have been described in rare forms of hereditary PD (Dauer and Przedborski, 2003). More recently, recessive mutations in the DJ-1 gene were proposed to play a role in cellular response to oxidative stress (Bonifati et al., 2003). However, none of these genes have been demonstrated to be mutated in sporadic PD, which constitutes more than 95% of total PD cases.

An approach to explore the etiology of PD is based on the study of changes in the up or down regulation of gene expression, which might increase the vulnerability of the neurons to cell death or even cause it. A number of studies have reported alterations in the expressions of various genes, such as decrease in calcium-binding protein (28 kDa calbindin-D) in the SN (Iacopino and Christakos, 1990) and D₃ receptor mRNA in lymphocytes (Nagai et al., 1996) from PD patients.

Among the neurotoxins used to induce parkinsonism in animals, N-methyl-4-phenyl-1,2,3,6-tetrahydropyridine (MPTP) best replicates many of the neurochemical and anatomical characteristics of the Parkinsonian syndrome in rodents, primates and other species (Dauer and Przedborski, 2003). In spite of certain limitations intrinsic to this model, namely lack of progressive nature and Lewy bodies (Inclusion bodies), much has been learned about the molecular events that lead to dopaminergic neurodegeneration.

Using the MPTP animal model alterations in the expression of several genes were observed. For example, an increase of glutamate decarboxylase mRNA was observed in a subpopulation of neurons in the putamen of parkinsonian monkeys, which provides further evidence that striato-pallidal GABAergic neurons are hyperactive in MPTP-treated parkinsonian monkeys (Soghomonian and Laprade, 1997). An increase in Bax (a cell death effector) mRNA expression in SN, with a concomitant increase in Bax immunoreactivity was also detected in the same model (Hassouna at al., 1996).

More recently, the MPTP model has been employed to assess differential gene expression changes in the midbrain of mice (Grunblatt et al., 2001), to obtain a more global picture of the series of events occurring during degeneration. The median density microarray employed in the study contained around 1200 genes. Thus, a limited picture of the differential gene expressions that may be altered was observed. Nevertheless, the study demonstrated alterations in genes related to oxidative-stress, inflammation, nitric oxide, glutamate excitotoxicity and neurotrophic factors pathways, which were up or down regulated. Additional gene pathways, not described previously, including cell cycle and iron metabolism regulation, apoptosis, intermediary metabolism and signal transduction have also been observed.

Furthermore, an increase in interleukin-1 beta (IL-1β) MRNA, in the pro-death genes BAD, BAX, and BID was reported in methamphetamine treated rats (Yamaguchi et al., 1991). Concomitantly, there were significant decreases in the anti-death genes Bcl-2 and Bcl-XL (Cadet et al., 2001; Jayanthi et al., 2001).

In another animal model for PD, caused by depletion of dopamine via 6-hydroxydopamine (6-OHDA), an increase in the glutamate decarboxylase mRNA expression was found in adult rats striatum as well as in neonates. A parallel increase in preproenkephalin and a decrease in preprodynorphin mRNA levels was observed (Laprade and Soghomonian, 1999).

Current accepted clinical criteria for the diagnosis of PD, such as Unified Parkinson's Disease Rating Scale (UPDRS) (Fahn, 1987), provide high sensitivity for detecting parkinsonism (Brooks, 1998). However, there are no sensitive and specific biochemical markers that can be used to reliably diagnose clinical and especially preclinical PD for sporadic and most familial PD cases.

One approach in the diagnosis of PD is functional imaging, which provides a means of discriminating typical from atypical PD, revealing characteristic patterns of loss of dopaminergic function. In addition, positron emission tomography (PET) and single photon emission tomography (SPECT) show preserved levels of striatal metabolism and dopamine receptor binding in PD, whereas levels are reduced in the atypical variants (Brooks,1998). Still these tools do not give an exact diagnosis of PD, and often, experts in PD changed their diagnoses infrequently during the 7.6-year follow-up (Jankovic et al., 2000).

Moreover, all these diagnostic methods are able to detect subjects with PD only after nearly 70% of the neurons have been degenerated, as only at this point symptoms appears (Zigmond et al., 1989). This, of course, makes treatment and maybe even rescue of the neurons nearly impossible. In view of this, it is desirable to diagnose the disease at an early stage and, for this reason, it is important to develop an early diagnostic method.

There are already many drugs that show neuroprotective effects in vitro and in vivo (Drukarch and van Muiswinkel, 2001; Grunblatt et al., 2001 and 2003; Koller, 2002; Olanow et al., 1996 and 1998; Riederer et al., 2000 and 2002; Soto-Otero et al., 2002). Additionally, gene therapy was shown to be most successful in delaying the neurodegenerative process (Azzouz et al., 2002; Le and Frim, 2002; Luo et al., 2002; McBride and Kordower, 2002; Monville, 2002; Olanow, 2002; Senior, 2002; Tenenbaum et al., 2002). The problem is that these drugs and methods did not show any success in patients with PD, because the beginning of therapy is too late and the number of surviving neurons is too small. Therefore, an early diagnosis may provide a better time point for the submission of therapeutic strategies that can protect against the cell death occurring in PD and to prevent the progress of the disease.

Therefore, it is crucial to treat PD patients before substantial damage of neurons occurs, and typical symptoms appear. Thus, there is a need for an early diagnostic method for detection and monitoring of PD at an early stage.

### SUMMARY OF THE INVENTION

The present invention relates, in one aspect, to the use of molecular markers for Parkinson's disease for the purpose of diagnosis, monitoring disease progression and effect of treatment.

In another aspect, the present invention relates to methods for diagnosis, prognosis and follow up of Parkinson's disease comprising the use of measurable biological markers, wherein said markers are one or more genes exhibiting an altered expression pattern, or gene products encoded thereby.

The genes for use as molecular markers according to the invention are genes which expression is upregulated or downregulated in PD patients, as detailed hereinafter.

### BRIEF DESCRIPTION OF THE FIGURES

FIG. 1 shows the relative expression levels of 137 genes differentially expressed in PD samples relative to control samples. Only genes that met the criteria of being altered by a factor of 1.5 relative to control and passed the Wilcoxon test at the significant level of p<0.05 were included. Genes are clustered by their relative expression levels over the 12 samples. Expression levels are color coded relative to the mean: green for values less than the mean and red for values greater than the mean.

FIGS. 2A-B show functional cluster analysis of genes involved in biological processes categorized according to Gene Ontology. Pie chart showing the distribution of down-regulated (2A) and up-regulated (2B) genes in PD samples compared to control brains. The number of altered genes in the different functional groups is indicated. Each gene was assigned a single category to avoid overestimation of the true size of each functional group. The total number of genes in (2A) is 68 and in (2B) is 69.

FIGS. 3A-3B show a heat map of differentially expressed genes that changed in at least five out of six PD samples by a factor equal or over 1.5. Patient sample signals were compared to the average of the control sample signals (geomean). Down-regulated (3A) and up-regulated (3B) genes in PD samples compared to control brains. Expression levels are color coded relative to the mean: green for values less than the mean and red for values greater than the mean.

FIGS. 4A-4L show a real-time quantitative Polymerase Chain Reaction (PCR) analysis confirmation of differentially expressed genes in PD. Following oligonucleotide hybridization, a selected number of genes whose expression was altered in PD SN, were validated in three separate brain areas, SNpc, SNr and the cerebellum, to detect tissue specific gene alterations. For SKP1A the values were adjusted to be higher than one and a log Y axis was applied. Standard curves represent relative gene expression normalized to the geometric mean of four house-keeping genes, as described in the Materials and Methods. Data points corresponding to PD and control cases are indicated. ANOVA, *p<0.05 vs. control.

FIGS. 5A-5B show an immunohistology analysis confirmation of differentially expressed SKP1 protein in the melanized dopaminergic neurons of the SNpc from parkinsonian brains. SKP1 immunoreactivity inside the melanin-containing dopamine (DA) neurons are indicated by blue inclusions (see green arrows). SKP1 is almost absent in PD.

### DETAILED DESCRIPTION OF THE INVENTION

The present invention relates to the use of specific molecular or biological markers for detection, prognosis and follow up of treatment of Parkinson's disease.

According to the present invention, genes with altered expression pattern in PD were found, such as for example the genes ALDH1A1, ARPP-21, HSPA8, SKP1A, SLC18A2, SRPK2, TMEFF1, TRIM36, ADH5, PSMA3, PSMA2, PSMA5, PSMC4, HIP2, PACE4, COX6A1, PFKP, OXCT, GBE1, UQCRC2, LANCL1, TRIP15, PIK3CA, PLCL1, GNG5, GNAI1, VEGF, RHOB, NR4A2, SCL3 1A2, SCP2, PIGH, ARIH2, GMPR2, PP, IKBKAP, PRKACB, PTPRN2, BCAS2, IARS, PPP1R8, SEP15, TAF9, ZFP103, WRB, TMEM4, SMARCA3, FMR1, PDE6D, SGCE, AUH, SLC16A7, ATP6V1E1, UGTREL1, SEC22L1, CD9, CDH19, DUSP1, HSA6591, ACTR3, KIF2, TUBB2, ASPA, HELO1, C3orf4, CBR1, XPOT, LOC51142, NY-REN-45, SET0-2, EGLN1, EIF4EBP2, LGALS9, LOC56920, LRP6, MAN2B1, PARVA, PENK, SELPLG, SPHK1, SRRM2, ZSIG11, ITGB3BP, ITGAM, COL18A1, TM4SF9, LAMB2, HS3ST2, TSTA3, COL5A3, PALM, MYOM1, FLNB, HMBS, KRT2A, CSK, NUDC, HYPE, GAK, SIAT1, CSF1R, ICSBP1, CD22, ERCC1, DNAJB5, TRAF3, MMP9, EIF4G1, RPL36, SRPK1, CSNK1G2, RPS6KA1, JIK, LNK, INPP5D, TCOF1, NAPG, SLC19A1, ITSN1, LOC51035, PMVK, C21orf2, EFEMP2, TBL1X, APRT, SPUF, GLTSCR2, ADIR, PSCD4, CBFA2T1, CUGBP1, ING4, STAT6, ZNF239, TAL1, TAF11, MXD4, RDHL, LOC51157, LRP6, MBD3, and C9orf7.

Thus, in one embodiment, the invention relates to the use of molecular markers for the detection, prognosis and follow up of treatment of Parkinson's disease, wherein the molecular markers are one or more of the above genes or their gene products.

It was further found by the inventors that some of the above mentioned genes show an increased expression in PD patients while other genes show a decreased expression, as compared to healthy individuals. Tables 3 and 4 hereinafter present the downregulated and upregulated genes, respectively, and their characterization including, for each gene, the Gene Bank number, the full gene name and its function.

In accordance to the present invention, the following genes were found to be upregulated, namely, to exhibit an increased level of expression in PD, and will be referred to hereinafter as “the upregulated genes”: EGLN1, EIF4EBP2, LGALS9, LOC56920, LRP6, MAN2B1, PARVA, PENK, SELPLG, SPHK1, SRRM2, ZSIG11, ITGB3BP, ITGAM, COL18A1, TM4SF9, LAMB2, HS3ST2, TSTA3, COL5A3, PALM, MYOM1, FLNB, HMBS, KRT2A, CSK, NUDC, HYPE, GAK, SIAT1, CSF1R, ICSBP1, CD22, ERCC1, DNAJB5, TRAF3, MMP9, EIF4G1, RPL36, SRPK1, CSNK1G2, RPS6KA1, JIK, LNK, INPP5D, TCOF1, NAPG, SLC19A1, ITSN1, LOC51035, PMVK, C21orf2, EFEMP2, TBL1X, APRT, SPUF, GLTSCR2, ADIR, PSCD4, CBFA2T1, CUGBP1, ING4, STAT6, ZNF239, TAL1, TAF11, MXD4, RDHL, LOC51157, LRP6, MBD3, and C9orf7.

Further in accordance with the present invention, the following genes were found to be downregulated, namely, to exhibit a decreased level of expression in PD, and will be referred to hereinafter as “the downregulated genes”: ALDH1A1, ARPP-21, HSPA8, SKP1A, SLC18A2, SRPK2, TMEFF1, TRIM36, ADH5, PSMA3, PSMA2, PSMA5, PSMC4, HIP2, PACE4, COX6A1, PFKP, OXCT, GBE1, UQCRC2, LANCL1, TRIP15, PIK3CA, PLCL1, GNG5, GNAI1, VEGF, RHOB, NR4A2, SCL31A2, SCP2, PIGH, ARIH2, GMPR2, PP, IKBKAP, PRKACB, PTPRN2, BCAS2, IARS, PPP1R8, SEP15, TAF9, ZFP103, WRB, TMEM4, SMARCA3, FMR1, PDE6D, SGCE, AUH, SLC16A7, ATP6V1E1, UGTREL1, SEC22L1, CD9, CDH19, DUSP1, HSA6591, ACTR3, KIF2, TUBB2, ASPA, HELO1, C3orf4, CBR1, XPOT, LOC51142, NY-REN-45, SET0-2.

As used herein the terms “molecular markers” and “biological markers” are used interchangeably to include gene products that can be identified and measured for diagnosing PD in samples obtained from individuals.

The term “gene products” as used herein refers to the expression product of a gene by conversion of the information contained in a gene into a gene product. A gene product can be the direct transcript of the gene, i.e., an RNA such as MRNA, tRNA, or any other type of RNA, or a protein produced by translation of a MRNA.

As used herein, the term “genes with altered expression pattern in PD” refers to genes that are upregulated or downregulated in PD patients in comparison, e,g., to healthy individuals. The term “upregulation” as used herein in the context of upregulation of a gene refers to any process which results in an increase in production of a gene product, e.g. RNA or protein expressed by the gene. The term “downregulation” as used herein in the context of downregulation of a gene refers to any process which results in a decrease in production of a gene product, e.g. RNA or protein expressed by the gene.

The level of gene expression, including the level of gene upregulation or downregulation, can be measured by known procedures as described hereinafter. In general, gene upregulation or downregulation comprises any detectable increase or decrease in the production of a gene product. The terms upregulated and gene activation can also mean that the observed activity relative to a baseline level is a statistically significant difference (i.e., increase or decrease). A difference is typically considered to be “statistically significant” if the probability of the observed difference occurring by chance (the p-value) is less than some predetermined level. As used herein a “statistically significant difference” refers to a p-value that is <0.05, preferably <0.02 for the downregulated genes (Table 3), and more preferably <0.005 for the upregulated genes (Table 4). In accordance with the present invention, the increase of expression in the upregulated genes is at least 1.5-fold, preferably 1-3 fold (Table 4) and the decrease of expression in the downregulated genes is at least 0.66-fold or below, preferably 0.3-0.66 (Table 3).

In one preferred embodiment of the invention, the biological markers for PD are one or more downregulated genes selected from the group consisting of ALDH1A1, ARPP-21, HSPA8, SKP1A, SLC18A2, SRPK2, TMEFF1, TRIM36, ADH5, PSMA3, PSMA2, PSMA5, PSMC4, and gene products thereof, and/or one or more upregulated genes selected from the group consisting of EGLN1, EIF4EBP2, LGALS9, LOC56920, LRP6, MAN2B1, PARVA, PENK, SELPLG, SPHK1, SRRM2, ZSIG11, and gene products thereof.

In one embodiment, the invention relates to a method for diagnosis, prognosis and/or follow up of treatment of Parkinson's disease comprising the use of molecular markers, wherein the molecular markers are one or more of the upregulated and/or downregulated genes of the invention. Preferably, the molecular markers are one or more downregulated genes selected from the group consisting of ALDH1A1, ARPP-21, HSPA8, SKP1A, SLC18A2, SRPK2, TMEFF1, TRIM36, ADH5, PSMA3, PSMA2, PSMA5, PSMC4, and gene products thereof, and/or one or more upregulated genes selected from the group consisting of EGLN1, EIF4EBP2, LGALS9, LOC56920, LRP6, MAN2B1, PARVA, PENK, SELPLG, SPHK1, SRRM2, ZSIG11, and gene products thereof.

The methods of the invention comprise obtaining a sample from the individual to be tested and detecting in the sample decreased or increased levels of one or more gene products specific to the upregulated and/or downregulated genes of the invention.

The tested individual may be an individual suspected of having PD, an individual exhibiting Parkinsonian-like symptoms, a de novo PD patient, a PD patient under treatment with a neuroprotective or another suitable PD drug, to follow up progress of the disease and/or to monitor effectiveness of the treatment. Thus, the invention provides methods for diagnosing PD at an early stage and for monitoring treatment of PD patients, both familial and sporadic PD patients.

Any suitable sample can be used for detection, but preferably blood, serum or biopsy samples of skin obtained from the tested individual are used.

A variety of different assays can be utilized to detect alteration in gene pattern expression, including methods that detect gene transcript levels and gene encoded protein levels. More specifically, the diagnostic and prognostic methods disclosed herein involve obtaining a sample from an individual and determining the expression level of one or more of the upregulated and/or downregulated genes in the sample. Usually this determined value or test value is compared against some type of reference such as the expression level in control (healthy individuals, non-PD patients) or a baseline value.

In accordance with the invention, also the use of the molecular markers in vivo is contemplated.

When the gene product to be tested is RNA extracted from the sample, the level of gene expression, i.e the level of gene upregulation or downregulation, can be measured by known procedures including, but not limited to, nucleic acid probe arrays, Northern blots, RNase protection assays (RPA), quantitative reverse-transcription PCR (RT-PCR), dot blot assays and in-situ hybridization.

In one preferred embodiment, nucleic acid probe arrays are used to detect and quantitate gene transcript (as described in the Examples below). The arrays can be of different types and may include probes of varying types such as, for example, short-length synthetic probes (20-mer or 25-mer), full length cDNA or fragments of gene), amplified DNA, fragments of DNA (generated by restriction enzymes, for example) and reverse transcribed DNA. The array may be a custom array, including probes that hybridize to particular preselected subsequences of mRNA gene sequences of the genes or amplification products thereof, or a generic array, designed to analyze mRNAs irrespective of sequence.

In the method using probe array, nucleic acids obtained from a test sample are usually reversed transcribed into labeled cDNA, although labeled mRNA can be used directly. The test sample containing the labeled nucleic acids is then contacted with the probes of the array and, after any labeled nucleic acid related to the tested genes present in the sample hybridizes to the probes, the array is typically subjected to one or more high stringency washes to remove unbound nucleic acids and to minimize nonspecific binding to the nucleic acid probes of the arrays. Binding of labeled nucleic acid is detected using any of a variety of commercially available scanners and accompanying software programs. For example, if the nucleic acids from the sample are labeled with fluorescent labels, hybridization intensity can be determined by, for example, a scanning confocal microscope in photon counting mode. The label can provide a signal that can be amplified by enzymatic methods or other labels can be used including, for example, radioisotopes, chromophores, magnetic particles and electron dense particles.

Those locations on the probe array that are hybridized to labeled nucleic acid are detected using a reader as commercially available. For customized arrays, the hybridization pattern can then be analyzed to determine the presence and/or relative amounts or absolute amounts of known mRNA species in samples being analyzed.

In another preferred embodiment, real time reverse-transcription PCR (real time RT-PCR) methods can be used to determine the quantity of the gene mRNA present in the sample (see Example below). These methods involve measuring the amount of amplification product formed during an amplification process, for example, by fluorogenic nuclease assays, to detect and quantitate specific transcripts of the genes of interest. These assays continuously measure PCR product accumulation using a dual-labeled fluorogenic oligonucleotide probe as in the approach frequently referred to in the literature simply as the TaqMan® method.

The probe used in real time PCR assays is typically a short (ca. 20-25 bases) polynucleotide that is labeled with two different fluorescent dyes: a reporter dye at the 5′ terminus of the probe and a quenching dye at the 3′ terminus, although the dyes can be attached at other locations on the probe as well. For measuring a specific transcript, the probe is designed to have at least substantial sequence complementarity with a probe binding site on the specific transcript. Upstream and downstream PCR primers that bind to regions that flank the specific transcript are also added to the reaction mixture for use in amplifying the nucleic acid.

When the probe is intact, energy transfer between the two fluorophores occurs and the quencher quenches emission from the reporter. During the extension phase of PCR, the probe is cleaved by the 5′ nuclease activity of a nucleic acid polymerase such as Taq polymerase, thereby releasing the reporter dye from the polynucleotide-quencher complex and resulting in an increase of reporter emission intensity that can be measured by an appropriate detection system. The fluorescence emissions created during the fluorogenic assay is measured by commercially available detectors that comprise computer software capable of recording the fluorescence intensity of reporter and quencher over the course of the amplification. These recorded values can then be used to calculate the increase in normalized reporter emission intensity on a continuous basis and ultimately quantify the amount of the mRNA being amplified.

In the assay based on dot blots and in-situ hybridization, a sample from an individual being tested for PD is spotted on a support, e.g., a filter, and then probed with labeled nucleic acid probes that specifically hybridize with nucleic acids derived from one or more of the upregulated or downregulated genes of the invention. After hybridization of the probes with the immobilized nucleic acids on the filter, unbound nucleic acids are rinsed away and the presence of hybridization complexes detected and quantitated on the basis of the amount of labeled probe bound to the filter.

Thus, according to the invention, gene expression pattern is determined by extraction of RNA from biological material, preferably blood or biopsy samples of skin. The RNA is isolated rapidly by a commercially available kit. The RNA is then tested through hybridization to a customized GeneChip array containing the selected genes and relevant house-keeping genes serving for normalization, or by means of real time PCR for each of the selected genes. The gene expression pattern is determined via comparison to the expression of positive and negative control RNA (with de-novo PD and healthy subjects, respectively). The pattern of the gene expression obtained via one of the techniques should be similar to the pattern described in Table 3 or 4 in order to define the subject as PD patient.

In another embodiment, the gene product obtained by expression of the upregulated or downregulated gene is a protein, that can be detected by an antibody, or a fragment thereof, able to bind to that protein. The antibody, or a fragment derivative, may be detectably labeled with any appropriate marker, for example, a radioisotope, an enzyme, a fluorescent label, a paramagnetic label, or a free radical.

In a preferred embodiment, the invention relates to a method for diagnosing occurrence of Parkinson's disease in an individual exhibiting Parkinsonian-like symptoms, comprising detecting in a sample obtained from said individual a decreased level of expression of one or more of the downregulated genes, more preferably one or more of the genes ALDH1A1, ARPP-21, HSPA8, SKP1A, SLC18A2, SRPK2, TMEFF1, TRIM36, ADH5, PSMA3, PSMA2, PSMA5, and PSMC4, and/or an increased level of expression of one or more of the upregulated genes, more preferably one or more of the genes EGLN1, EIF4EBP2, LGALS9, LOC56920, LRP6, MAN2B1, PARVA, PENK, SELPLG, SPKH1, SRRM2, and ZSIG11. An increase of the expression of one or more of the upregulated genes by at least about 1.5-fold and/or a decrease of the expression of one or more of the downregulated genes to about 0.66-fold or below will indicate that the individual suffers from PD.

In comparison with the current methods used for diagnosis of PD, this method has the advantage not to use rare genetically mutations or familial history of the disease, but rather use general gene expression changes which occur also in sporadic PD. These gene expression alterations may be caused not only as a consequence of specific genetic background, but also as a consequence of environmental background. Therefore, this method will detect both PD patients carrying gene mutations and sporadic PD patients very early in the development of the disease, before extensive cell death and irreversible substantial damage of neurons occurs.

When untreated, PD progresses to total disability, often accompanied by general deterioration of all brain functions, and may lead to an early death. Treated, the disorder still impairs people in varying ways. Most people respond to some extent to medications. The extent of symptom relief, and how long this control of symptoms lasts, is highly variable.

Thus, in another embodiment, the invention provides a method for prognosticating or monitoring the treatment of a PD patient, which comprises detecting in a sample obtained from said patient the level of expression of one or more of the dowregulated or upregulated genes of the invention, whereby an increased level of expression of one or more of the downregulated genes, more preferably of one or more of the genes ALDH1A1, ARPP-21, HSPA8, SKP1A, SLC18A2, SRPK2, TMEFF1, TRIM36, ADH5, PSMA3, PSMA2, PSMA5, and PSMC4, and/or a decreased level of expression of one or more of the upregulated genes, more preferably one or more of the genes EGLN1, EIF4EBP2, LGALS9, LOC56920, LRP6, MAN2B1, PARVA, PENK, SELPLG, SPHK1, SRRM2, and ZSIG11, indicate the effectiveness of the treatment.

As shown in the Examples below, the inventors identified global major differentially expressed genes in the most affected brain area in PD, the substantia nigra pars compacta (See Tables 3 and 4). Employing Affymetrix high density DNA microarray, differentially expressed genes compared to aged-matched controls were identified. The cerebellum of PD, an unaffected brain region, served as control for tissue specificity. Confirmation of gene expressions was achieved by analysis with quantitative real-time PCR.

It was found that the downregulated genes belong to signal transduction, protein degradation (e.g. ubiquitin-proteasome subunits), dopaminergic transmission/metabolism, ion transport, protein modification/phosphorylation and energy pathways/glycolysis functional classes. The upregulated genes clustered mainly in biological processes involving cell adhesion/cytoskeleton, extracellular matrix components, cell cycle, protein modification/phosphorylation, protein metabolism, transcription and inflammation/stress (e.g. key iron and oxygen sensor EGLN1).

One major finding was the particular decreased expression of SKP1A, a member of the SCF (E3) ligase complex specifically in the substantia nigra (SN) of sporadic parkinsonian patients. SKP1A decline was accompanied by decreased expression in various subunits of the 26S proteasome, in energy pathways and signal transduction, in parallel with a prominent increase in a number of genes with functional activities related to cell adhesion/cytoskeleton, extracellular matrix components and inflammation/stress.

SKP1 is part of the Rbx family of RING proteins (Kamura et al., 1999) functioning within modular multiprotein Skp1, Cullin and a substrate-recognizing F-box protein (SCF). This unit allows the formation of multiple E3 complexes, which, in turn, are able to recognize a wide spectrum of different protein substrates.

SCF complexes are modular: SKP1 can interact with several F-box proteins, which are responsible for specific target recognition, thereby providing functional diversity and increasing the repertoire of proteins processed by this complex. Humans express only one functional SKP 1 isoform (Semple, 2003). Thus, the decrease in its expression observed, may constitute a rate limiting factor and may account for the accumulation of a wide spectrum of ubiquitinated protein aggregates in brains of PD patients such as tyrosine hydroxylase, synphilin-1, alpha-synuclein, phosphorylated tau (Liani et al., 2004; Meredith et al., 2004; Zhang and Goodlett, 2004).

Quantitative real time PCR analysis revealed that the SN expresses extremely low mRNA levels of SKP1A in both normal and PD subjects. For example, control samples display very low expression values (0.013±0.011), differing by more than two orders of magnitude from Rab3B (1.80±0.70), whose expression was not affected in SNpc of PD. A clear trend of decreased expression of SKPA1A in the SNpc of PD was observed. The expression of SKP1A was not affected in the SNr or in cerebellum of PD.

In addition to this selective decrement in SKP1A, a concomitant decrease in the 20S proteasome subunits alpha-5 (PSMA5), alpha-3 (PSMA3) and alpha-2 (PSMA2) mRNAs and in two subunits of the 19S regulatory complex of the 26S proteasome, the non-ATPase subunit 8 (PSMD8/Rpn12) and the ATPase subunit 4 (PSMC4/TBP7/Rpt3), was observed in the SN of PD brains, which may further contribute to dopaminergic neuronal damage in PD.

These findings reveal novel players in the neurodegenerative scenario and provide potential targets for the development of novel drug compounds.

The present invention thus further provides a method for screening for an agent useful for treating Parkinson's disease, which comprises identifying an agent that upregulates the expression of one or more of the downregulated genes, preferably the downregulated genes ALDH1A1, ARPP-21, HSPA8, SKP1A, SLC18A2, SRPK2, TMEFF1, TRIM36, ADH5, PSMA3, PSMA2, PSMA5, PSMC4, and/or downregulates the level of expression of one or more of the upregulated genes, preferably EGLN1, EIF4EBP2, LGALS9, LOC56920, LRP6, MAN2B1, PARVA, PENK, SELPLG, SPHK1, SRRM2, ZSIG11.

High throughput screening methods as known in the art can be used to identify compounds that will be useful for treatment of PD.

The invention will now be illustrated by the following non-limiting examples.

### EXAMPLES

Materials and Methods

(i) Human Brain Tissue

Post-mortem human brains were from the National German Brain-Net Center (Project no GA10) and the Netherlands Brain-Bank (Project no. 350). PD tissues were obtained from moderately to severe parkinsonism individuals based on the Hoelin & Yahr criteria in accordance with established guidelines (Hoehn and Yahr, 1967). All the subjects were negative for AD pathology according to Braak & Braak (Braak and Braak, 1997) (for details see Table 1). The average age for PD and control is 76.6 and 77.8 years, respectively. The average post-mortem delay (PMD, time interval from death to brain-freezing at −80° C.) for PD and control is 26.2 and 19.8 hours, respectively. At autopsy, brains were dissected; one half was snap frozen in liquid nitrogen as slices and the other half was stored in neutral buffered formalin for histopathology. Stringent criteria were used in the case selection of human brain tissues employed in these studies. Human SN and cerebellum regions were obtained from control and PD donors. All procedures were in accordance with the with consensus criteria established by the German and Netherlands Brain-Bank systems and in accordance NIH Guide for the Care and Use of Laboratory Human Tissue and approved by the University of Wutrzburg Ethics Committee (Würzburg, Germany).

(ii) Total RNA Extraction

Total RNA was prepared with a phenol-guanidine isothiocyanate reagent (peqGOLD TriFast; PeQLabGinbbH, Erlangen, Germany) (Lukiw and Bazan, 1997). RNA isolation reagents were prepared with 0.2 μM filtered diethyl pyrocarbonate (DEPC)-treated water (Fermentas Inc., Hanover, Md., USA) and used throughout the isolation procedure. Total RNA samples were spectrophotometrically scanned from 220 to 320 nm; the A260/A280 of total RNA was typically >1.9. In addition, formaldehyde agarose gel electrophoresis was conducted for quality control of total RNA. For all total RNAs extracted, the 28S/18S ratio was >1.5. Importantly, no significant differences in the spectral purity, rate of degradation, molecular size, or yield of SN and cerebellum total RNA between PD and control, were noted. Total RNAs was subjected to DNase-I digestion to get rid of genomic DNA residues and subsequently cleaned by the RNeasy Mini Kit (RNeasy Mini Kit; Qiagen Inc., Valencia, Calif., USA).

(iii) Array Processing

All experiments were performed using Affymetrix HG-FOCUS oligonucleotide arrays, as described at //www.affymetrix.com/support/technical/datasheets/human_datasheet.pdf. Total RNA from each sample was used to prepare biotinylated target RNA, with minor modifications from the manufacturer's recommendations (http://www.affymetrix.com/support/technical/manual/expression_manual.affx). Briefly, 10 μg of mRNA was used to generate first-strand cDNA by using a T7-linked oligo(dT) primer. After second-strand synthesis, in vitro transcription was performed with biotinylated UTP and CTP (Enzo Diagnostics), resulting in approximately 100-fold amplification of RNA. A complete description of procedures is available at: http://bioinf.picr.man.ac.uk/mbcf/downloads/GeneChip_Target_Prep_Protocol_CR UK_v_{—}2.pdf. The target cDNA generated from each sample was processed as per manufacturer's recommendation using an Affymetrix GeneChip Instrument System (http://www.affymetrix.com/support/technical/manual/expression_manual.affx). Briefly, spike controls were added to 15 μg fragmented cDNA before overnight hybridisation. Arrays were then washed and stained with streptavidin-phycoerythrin, before being scanned on an Affymetrix GeneChip scanner. A complete description of these procedures is available at: http://bioinf.picr.man.ac.uk/mbcf/downloads/GeneChip_Hyb_Wash_Scan_Protocol_v_{—}2_web.pdf. Additionally, quality and amount of starting RNA was confirmed using an agarose gel. After scanning, array images were assessed by eye to confirm scanner alignment and the absence of significant bubbles or scratches on the chip surface. BioB spike controls were found to be present on all the arrays, with BioC, BioD and CreX also present in increasing intensity. A total of 17 gene chips were employed (12 for the SNpc and 5 for the cerebellum of PD patients and age-matched controls).

(iv) Statistical Analysis

Genes were filtered using Mas 5 algorithm results. One expected difficulty when working with post mortem samples is the various degrees of degradation in the RNA preparations. Therefore, cRNA originating from these samples contain more 3′ ends than 5′ prime ends. Since the Affymetrix array probe sets are designed so that the 3′ ends are selected whenever possible (http://www.affymetrix.com/support/technical/technotes/hgu133_design_technote.pdf), the analysis of relatively high-3′content samples becomes feasible. Probe sets detected by MAS 5 as Present (P) have a signal with a p-value lower than 0.04 (determined from the probes contained in the probe set). This allows us to use present call with confidence.

Samples that are partially degraded may hybridize to part of the probes in the probe sets leading to a high p-value (and detected as absent). If the probe set in at least four out of six samples was detected as present and all of the signals were greater than 20, we determined this probe set to be present. A list of 3517 probe sets representing genes with signals higher than 20 and detected as present (P) in all control samples, or with signals higher than 20 in all PD samples and detected as present in four out of the six samples was generated from the 8763 probe sets contained on the array (supplement http://eng.sheba.co.il/genomics).

262 probe sets differentiated between patient and control samples as determined by the Wilcoxon rank sum test (p-value<0.05) (supplement http://eng.sheba.co.il/genomics). Probe sets were further filtered selecting those where the ratio of the average (geomean) signals of the PD samples and the average signals of the control samples exceeded 1.5 or was lower than 0.66. The Bonferroni's correction has not been applied because it would result in loss of most of the valid hits. However the use of the aforementioned cut-offs together with the statistical test provide a high stringency analysis and the possibility to focus on defined subset of genes engaged in a common biological process(s). 69 probe sets were up regulated and 68 were down regulated.

(v) Real-Time Quantitative PCR Validation of Results

In order to validate the microarray results, we performed quantitative real-time RT-PCR for mRNA samples from PD and controls in the SNpc, pars reticulate (SNr) and cerebellum. Total RNA (1-0.4 mg) from each sample was reverse transcribed with random hexamer and oligodT primers using iScript™ cDNA Synthesis Kit (BioRad Laboratories, Hercules, Calif., USA, 170-8890). The genes were normalized to the house-keeping genes: beta actin (ACTB), ribosomal protein L13a (RPL13A), aminolevulinate delta synthase 1 (ALAS1) and glyceraldehyde-3-phosphate dehydrogenase (GAPDH) (QuantiTect™ Gene Expression Assay, Qiagen Inc., Valencia, Calif., USA, Hs ACTB Assay 241013 & Hs GAPD Assay 241011), chosen after analysis according to the program geNorm (vs. 3.3; to be download from: http://medgen31.ugent.be/jvdesomp/genorm/) (Vandesompele et al., 2002; Schulz et al., 2004). The geNorm program determined the most stable house-keeping genes from a set of tested genes in a given cDNA sample panel and calculates a gene expression normalization factor for each tissue sample based on the geometric mean of a user-defined number of house keeping genes. Originally, we tested a total of six house-keeping genes, including ribosomal 18S and cyclophilin A, but the latter two were found not suitable.

These house-keeping genes were tested for their stability and found be the most accurate for normalization. Absence of DNA contamination was verified by amplifying the house-keeping gene ribosomal 18S, and running the reaction solution on agarose gel to confirm the absence of product. Minus RT samples tested simultaneously with experimental samples by quantitative RT-PCR with FAM consistently yielded no amplification below 35 cycles using the above protocol. Real-time PCR was performed in the iCycler iQ system (BioRad Co., Hercules, Calif., USA) as described previously (Svaren et al., 2000; Ugozzoli et al., 2002). Briefly, 30-100 ng of cDNA and gene specific primers and probes produced by QuantiTect™ Custome Assay (Qiagen Inc., Valencia, Calif., USA,) (Table 2) were added to QuantiTect Probe PCR Master Mix (Qiagen Inc., Valencia, Calif., USA, 204343). Real-time PCR was subjected to PCR amplification (1 cycle at 95° C. for 15 min, 30-45 cycles at 94° C. C for 15 s, annealing and detecting with FAM at 56° C. for 30 s and extension at 76° C. for 30 s). All PCR reactions were run in duplicate. The amplified transcripts were quantified using the comparative CT method analyzed with the BioRad iCycler iQ system program. Standard curves for each amplification product were generated from 10-fold dilutions of pooled cDNA amplicons, isolated from agarose gel using MinElute™ Gel Extraction Kit (Qiagen Inc., Valencia, Calif., USA), to determine primer efficiency and quantization. Data was analyzed with Microsoft Excel 2000 to generate raw expression values. The differences in gene expression in the different brain regions were compared using the analysis of variance (ANOVA), the StatView software program (Stat View 5.0. software, SAS Institute Inc. Cary, N.C., USA) on a PC computer.

vi) Immunohistochemical Analysis

Paraffin-embedded substantia nigra from parkinsonian patients and age-matched controls underwent serial coronal sections (6 micrometer thickness) by microtome. Deparaffinized sections were unmasked in digital decloacking chamber (Biockare Medical, Walnut Creek, Calif., USA) using citrate buffer (pH 6.8), blocked with 10% normal goat serum for 2 h at RT and incubated overnight at 4° C., with anti-SKP1. Detection was obtained by appropriate biotinylated second antibody with streptavidin-peroxidase conjugate and S-(2-aminoethyl)-L-cysteine (AEC) as substrate, using Histostain-Plus kit (Zymed, South San Francisco Calif., USA) according to manufacture instructions, Counterstaining was performed with hematoxylin.

### Example 1

### Functional Classification of Differentially Expressed Genes in PD Patients

DNA microarray analysis was performed on postmortem SNpc from 6 parkinsonian patients and 6 aged-matched controls to identify alteration in gene transcription associated with the disease.

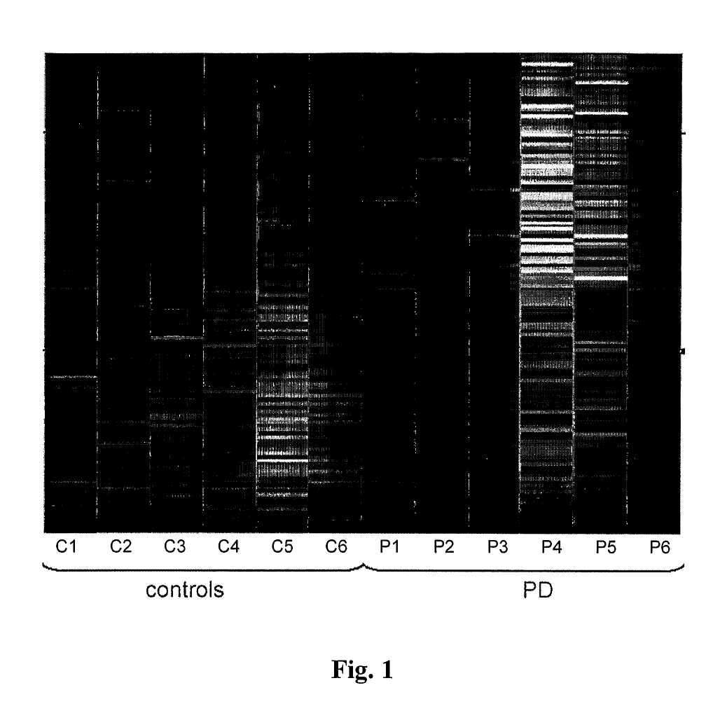

We confined our analysis to genes with at least a 1.5 fold differential expression and significance level of p<0.05. Of a total of 3517 valid probe sets analyzed (see Materials and Methods), 137 met the criteria. A heat map was performed depicting the relative expression levels of these 137 genes in PD and control samples (FIG. 1), of which 68 were transcriptionally down-regulated (Table 3) and 69 were up-regulated (Table 4).

Genes are clustered by their relative expression levels over the 12 samples. For negative control, five oligonucleotide hybridizations were performed in a non-related brain area, the cerebellum of both PD (2 samples) and control (3 samples), to assess tissue specificity of gene changes. Hierarchical clustering showed that both PD and normal cerebellum display a complete different pattern of expression (data not shown).

Afterwards, the genes were classified into functional groups according to GeneOntology annotation tools (Dennis et al., 2003) (http://apps1.niaid.nih.gov/David/upload.asp). The Program allows finding regulation trends in group of genes organized according to biological processes, molecular function or cellular components, as defined by the Gene Ontology Consortium (http://www.geneontology.org).

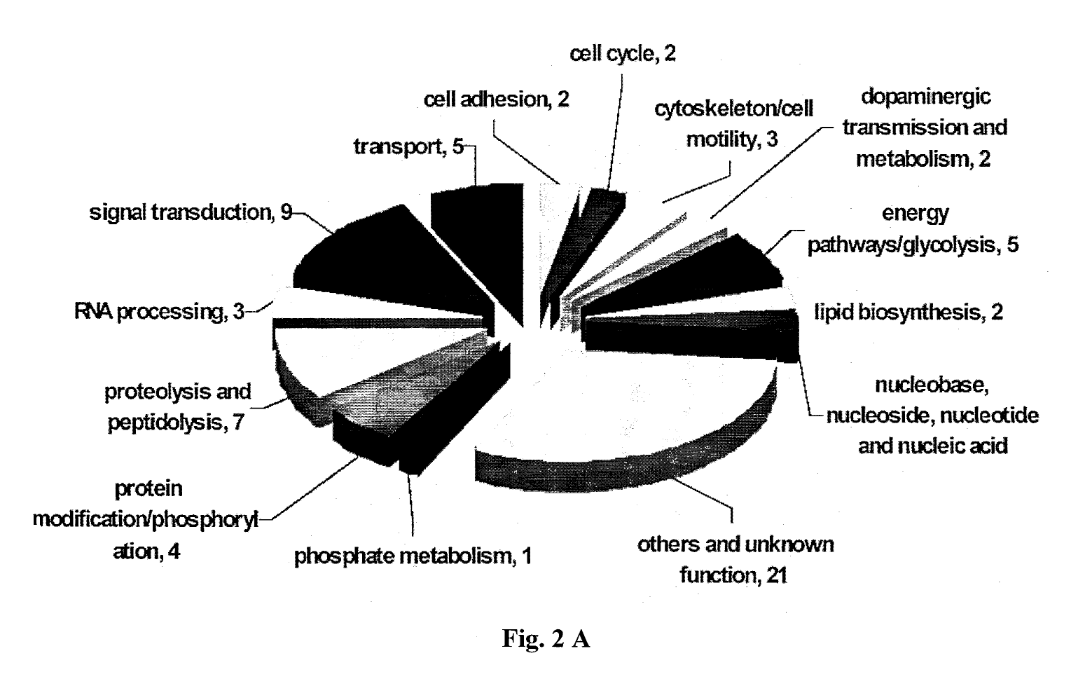

It is recognized that a given gene may be assigned to more than one function or biological pathway. This may result in overestimation of the true size of each functional group. We overcame this by assignment to each gene a single functional class. As shown in FIG. 2A, major gene downregulations were observed in the signal transduction, protein degradation, dopaminergic transmission and metabolism, ion transport, protein modification/phosphorylation and energy pathways/glycolysis functional classes in PD.

With respect to differentially upregulated genes in PD, they clustered mainly in biological processes involving cell adhesion/cytoskeleton, extracellular matrix components, cell cycle, protein modification/phosphorylation, protein metabolism, transcription and inflammation/stress (FIG. 2B).

Statistical analysis of gene categories, to identify genes that are the most over-represented or enriched, was accomplished with the EASE program (Hosack et al., 2003). Such groupings increase confidence in the results when the proportion of genes that change within a functional group is significantly greater than the fraction of such genes in the whole chip.

Functional classification with “Ease score” lower than 0.05 were marked as over represented. This analysis revealed that the most prominent changes in the differentially downregulated genes occurred in proteolysis and peptidolysis (10%) biological process, with an EASE score of p<0.02 (Table 3, marked by an asterisk) and in molecular functions related to ion transporter and hydrolase activities (p<0.05, data not presented). Within this proteolytic group we observed decreased expression of both catalytic and regulatory subunits of the UPS such as 20S proteasome subunits alpha-5 (PSMA5), alpha-3 (PSMA3) and alpha-2 (PSMA2), two subunits of the 19S regulatory complex of the 26S proteasome, the non-ATPase subunit 8 (PSMD8/Rpn12) and the ATPase subunit 4 (PSMC4/TBP7/Rpt3). PSMD8 slightly exceeded the upper limit of significance, but a clear trend of reduction was seen in the six patients (three of them >1.5 fold of control). Since PSMD8 clusters together with the other UPS components, we considered it as functionally affected in PD.

On the other hand, statistical significant trends in biological processes and molecular functions with increased gene expression in PD were found for cell adhesion/cellular matrix (˜15%) with an EASE score of p<0.005 (Table 4, marked by an asterisk) and for structural molecule and protein kinase activities (p<0.05, data not shown).

In parallel, an exceptionally stringent analysis was done selecting those genes that display a 1.5 fold differential expression in at least five out of the six parkinsonian SN. Each patient sample signals were compared to the mean of the control sample signals (geomean). This evaluation is of major importance, since it allows identifying variations in gene expression within each of the post-mortem tissue samples that may derive from a combination of factors such as age, cause of death, gender, post-mortem interval, severity of the disease, etc.

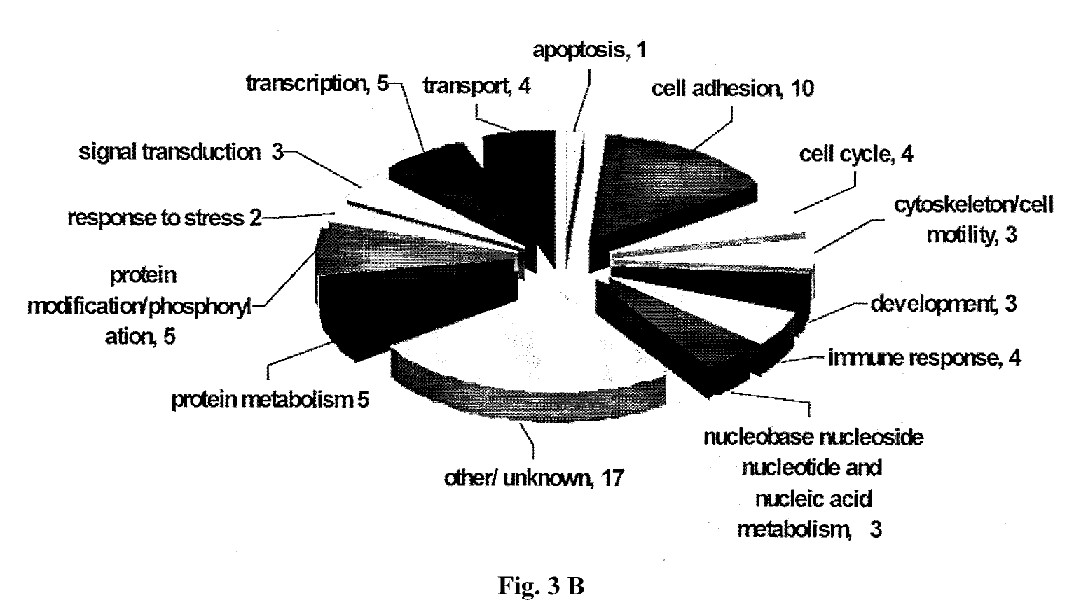

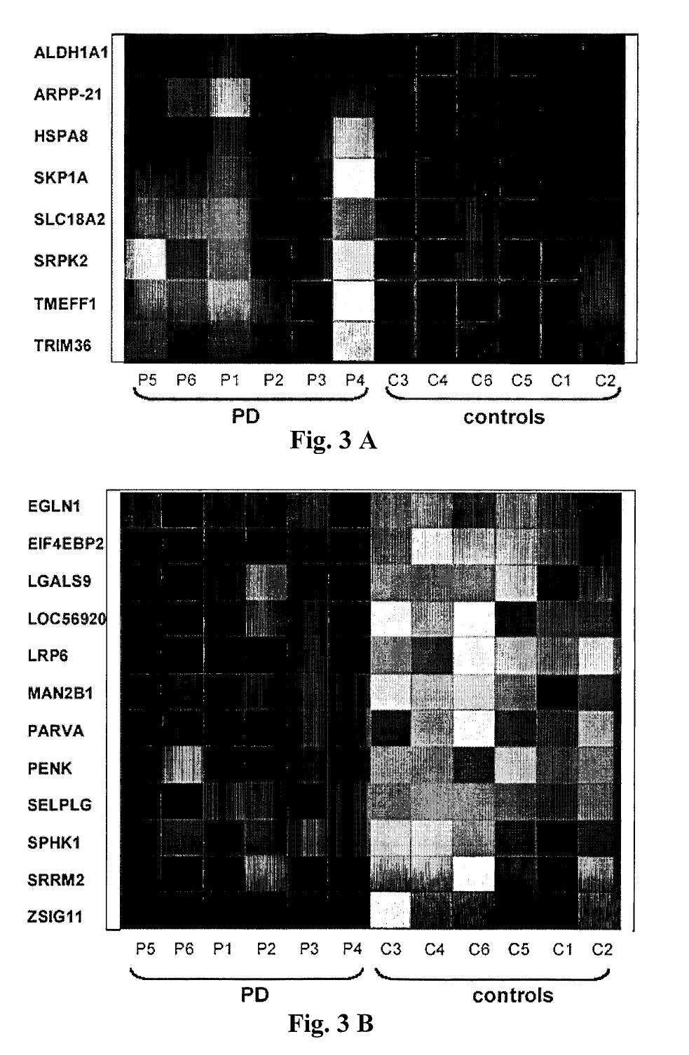

This approach restricted the total differentially expressed genes in the PD cases to only 20, of which 8 were decreased and 12 were increased respective to the control group (FIG. 3).

Importantly, of the 8 downregulated genes, 3 (37.5%) belong to DA neurotransmission and metabolism (FIG. 3A). These include cyclic AMP-regulated phosphoprotein (ARPP-21), solute carrier family 18 (vesicular monoamine member 2, VMAT2) and aldehyde dehydrogenase 1 family, member A1 (ALDH1A1). Two more genes are related to protein handling and degradation (S-phase kinaseassociated protein 1A (p19A), SKP1A and the chaperone heat shock 70 kDa protein 8, HSPA8). The last 3 genes are the SFRS protein kinase 2 (SRPK2), which participates in phosphorylation and protein modification processes, the tripartite motif-containing 36 (TRIM36) and the transmembrane protein with EGF-like and two follistatin-like domains 1 (TMEFF1), both of unknown function.

The 12 upregulated genes (FIG. 3B) include: parvin, alpha (PARVA), lectin, galactoside-binding, soluble, 9 (galectin 9) (LGALS9), and selectin P ligand (SELPLG), belonging to the cell adhesion functional group; proenkephalin (PENK) and low density lipoprotein receptor-related protein (6LRP6), which are related to the cell signaling class; egl nine homolog 1 (C. elegans) (EGLN1), eukaryotic translation initiation factor 4E binding protein 2 (EIF4BP2), mannosidase, alpha, class 2B, member 1 (MAN2B1) and sphingosine kinase 1 (SPHK1) are from the protein and lipid metabolism and phosphorylation categories; semaphorin sem2 (LOC56920), involved in cell development, putative secreted protein ZSIG11 (ZSIG11) and a serine/arginine repetitive matrix 2 (SRRM2), both of unknown functions.

### Example 2

### Quantitative Real-Time PCR

To confirm our findings of decreased expression of one of the major genes SKP1A, playing a key role in the UPS, we conducted real-time quantitative PCR for it and for two additional genes, HSC70 and VMAT2, all of them affected in at least five out of six parkinsonian patients by a factor <0.67.

We added two more samples to the experimental groups (Control 7 and Patient 7) in cases where the RNA used for the array hybridization was not adequate for the PCR. We analyzed three separate areas: the SNpc, SNr and the cerebellum. Cerebellum was chosen as an area not related to PD, for assessing specificity of gene changes.

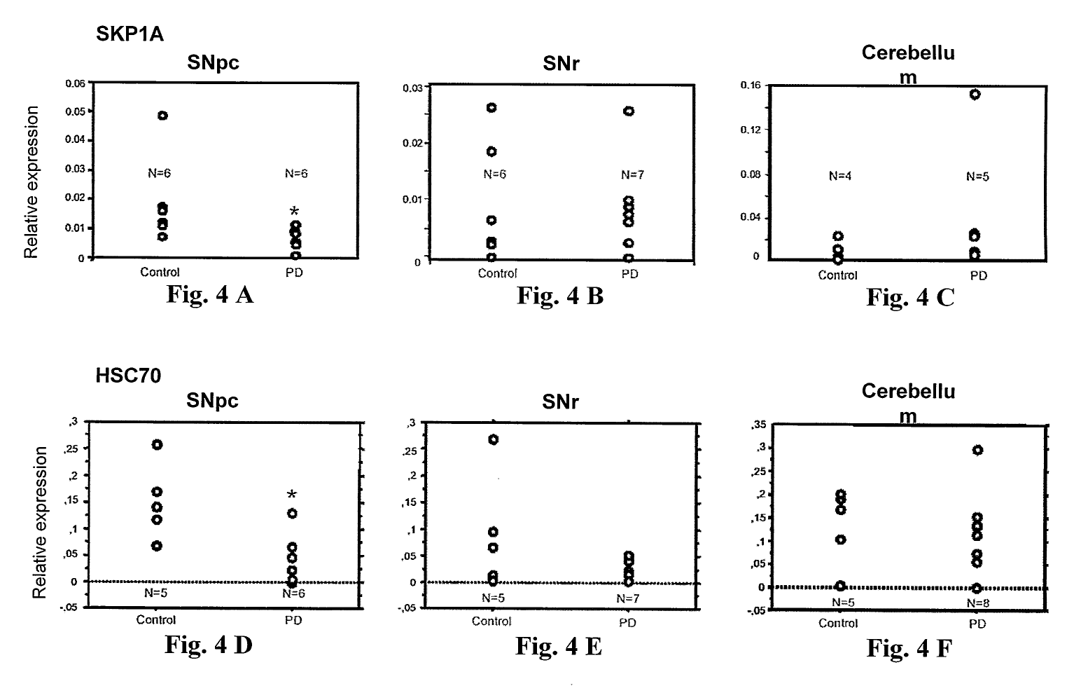

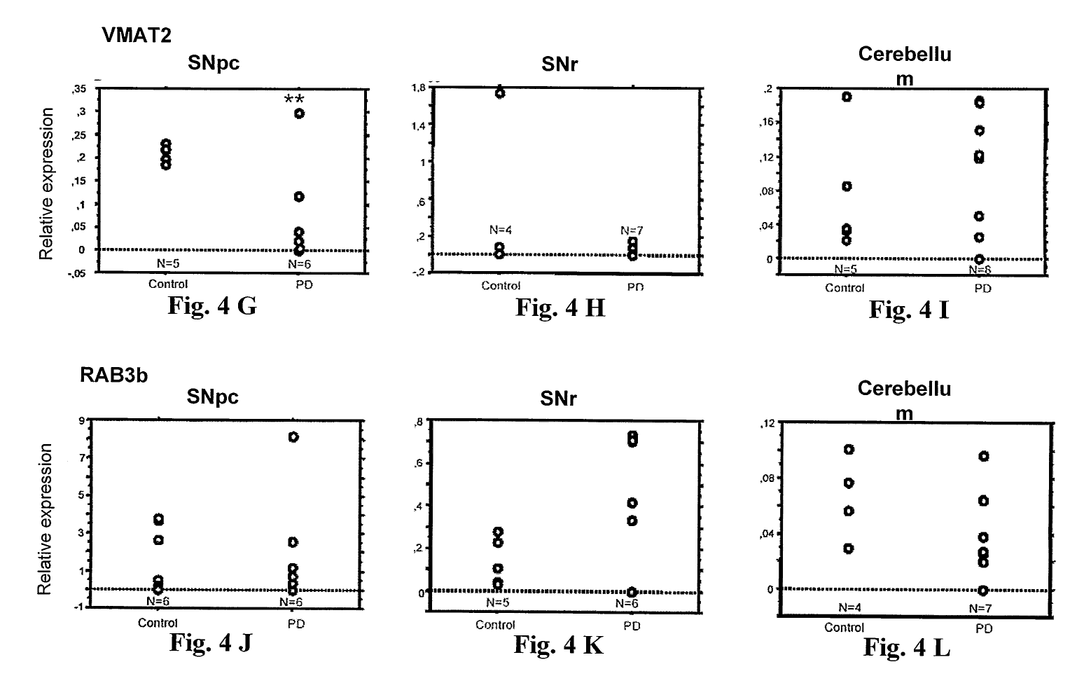

Real-time PCR analysis revealed that the SN expresses extremely low mRNA levels of SKP1A in both normal and PD subjects (FIG. 4). Although not statistically significant, a clear trend of decreased expression in the SNpc of PD was observed (FIG. 4A). The expression of SKP1A was not affected in the SNr or in cerebellum of PD (FIGS. 4B,C).

We confirmed significant decreased gene expression of HSP8, coding for HSC70/HSC54, both in the SNpc and SNr of parkinsonian patients (FIGS. 4D,E), whereas no significant alterations were observed in cerebellum from PD and controls (FIG. 4F). HSC70/HSC54 exists in two isoforms, the truncated one lacking 153 amino acids residues in the protein binding and variable domain (Tsukahara et al., 2000). The probe sets in the Affymetrix chip cannot discriminate between them, so the results from both the array hybridization and PCR analyses should represent a combined level of both types.

In addition to these novel findings, our array analysis also confirmed evidence for decreased expression of the vesicular DA transporter (VMAT2). Real-time PCR revealed a specific reduction of VMAT2 mRNA in the SNpc while the changes in the SNr were significantly less pronounced (FIGS. 4G,H). These alterations were specific for the SN since no significant difference was seen in the cerebellum between the PD and control samples (FIG. 4I).

To determine whether the reduced expressions found in both the arrays and in real-time PCR analysis was merely a reflection of a general reduction in mRNA levels in PD SN, or loss of synapses, we investigated the expression of three genes: Rab3b GTPase (FIGS. 4J,K,L), syntaxin 6 and the coatomer protein complex subunit zeta 2 (COP zeta 2) (data not presented), which are regulators of vesicle trafficking from endoplasmic reticulum to Golgi, vesicle membrane docking and neuronal exocytosis of hormones and neurotransmitters (Gonzalez and Scheller, 1999; Futatsumori et al., 2000; Wendler and Tooze, 2001). Their expression did not differ between PD and control samples in the different brain areas.

The stringent analysis performed in the experiments above, focusing on selected biological meaningful pathways in PD and the substantial agreement of gene changes reported in literature and confirmed by real-time PCR analysis. make the analysis of the present results valid.

### Example 3

### Detection of SKP1 Protein in the Melanized Dopaminergic Neurons of the SNpc from Parkinsonian and Healthy Control Brains

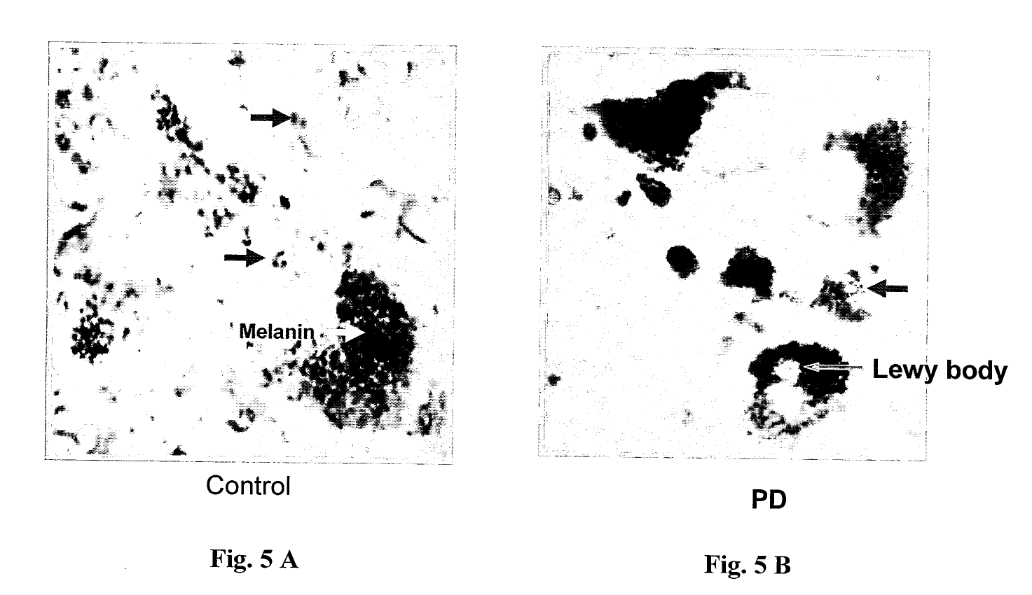

The immunohistology analysis of SKP 1 protein was carried out in samples of melanized dopaminergic neurons of the SNpc from parkinsonian brain and healthy control (as described in Materials and Methods). The results summarized in FIG. 5 show that SKP1 protein is present in normal samples but is almost absent in PD samples. Thus, the results obtained in Examples 1 and 2 above for MRNA expression of SKP1, were confirmed at the protein level by immunohistology analysis of SKP1 protein.

Summary of the Results

The results of the present invention show, for the first time, the global gene expression changes in the SNpc of post-mortem parkinsonian brains as compared to aged-matched controls. One major novel finding is the negative regulation of an essential component of protein catabolism, the SKP1A gene. Its decline was accompanied. by decreased expression in various subunits of the 26S proteasome, in energy pathways and signal transduction, in parallel with a prominent increase in a number of genes with functional activities related to cell adhesion/cytoskeleton, extracellular matrix components and inflammation/stress.

Reduction in SKP1A, a Component of the SCF Ubiquitin Ligase Multiprotein Complex, in Parkinsonian SNpc

SKP1 is part of the Rbx family of RING proteins (Kamura et al., 1999) functioning within modular multiprotein Skp1, Cullin and a substrate-recognizing F-box protein (SCF). This unit allows the formation of multiple E3 complexes, which, in turn, are able to recognize a wide spectrum of different protein substrates. SCF complexes are modular: SKP1 can interact with several F-box proteins, which are responsible for specific target recognition, thereby providing functional diversity and increasing the repertoire of proteins processed by this complex. Humans express only one functional SKP1 isoform (Semple, 2003). Thus, the decrease in its expression observed herein, may constitute a rate-limiting factor and may account for the accumulation of a wide spectrum of ubiquitinated protein aggregates in brains of PD patients such as tyrosine hydroxylase, synphilin-1, α-synuclein, phosphorylated tau (Liani et al., 2004; Meredith et al., 2004; Zhang and Goodlett, 2004).

Quantitative real time PCR analysis revealed that the SN expresses extremely low mRNA levels of SKP1A in both normal and PD subjects. For example, control samples display very low expression values (0.013±0.011), differing by more than two orders of magnitude from Rab3B (1.80±0.70), whose expression was not affected in SNpc of PD. It is also probable that the mRNA of SKP1A is particularly unstable and it degrades fast in post-mortem tissue. This may explain the great variation in the expression levels of the samples, although a clear trend of decreased expression in the SNpc of PD was observed. Because of the special design of the Affymetrix chip, even samples that are partially degraded, such as in post-mortem, may in part hybridize to the probes in the probe sets leading to a high p-value and considered as present. Thus, the chip strategy together with the stringent analysis (p<0.05, decrease in five patients by a factor of at least 1.5), make this result confident. The low SKP1A expression levels may explain, at least in part, the particular susceptibility of the SNpc to different types of stresses, such as increased iron concentration, enzymatic (monoamine oxidase) and non-enzymatic (auto-oxidation) DA metabolism, abnormal protein accumulation, proteasomal inhibition and neurotoxin-induced OS. In this context, the PA28 multisubunit proteasome activator (a component of the 26S proteasome) protein levels were shown to be very low in the SNpc of both normal and idiopatic PD subjects compared to other brain areas (McNaught et al., 2003), possibly exacerbating the already compromised DA-containing neurons to the various stress insults. The expression of SKP1A was not affected in the SNr or in cerebellum of PD.

A recent study has demonstrated that Parkin can also function within a novel SCF-like complex, along with the F-box/WD repeat protein hSel-10, responsible for substrate recognition, and Cullin1, which however does not include SKP1 (Staropoli et al., 2003). According to this finding, it is possible that Parkin associates with adaptor proteins other than hSel-10. Such complexes would likely display diverse substrate specificity and may explain the several targets that have been reported for Parkin (Dev et al., 2003).

Impairment in Proteasome Subunits

In addition to this selective decrement in SKP1A, a concomitant decrease in the 20S proteasome subunits alpha-5 (PSMA5), alpha-3 (PSMA3) and alpha-2 (PSMA2) mRNAs and in two subunits of the 19S regulatory complex of the 26S proteasome, the non-ATPase subunit 8 (PSMD8/Rpn12) and the ATPase subunit 4 (PSMC4/TBP7/Rpt3), was observed in the SN of PD brains, which may further contribute to dopaminergic neuronal damage in PD. The 19S complexes are located at one or both extremities of the 20S proteolytic core of the proteasome (Coux et al., 1996; Voges et al., 1999) and comprise at least 18 subunits (Glickman and Ciechanover, 2002). They are subdivided into two subcomplexes, the “base” and the “lid” which form the portions proximal and distal to the 20S proteolytic core respectively, and all together form the 26S complex. The lid consists of eight regulatory particle non-ATPase (Rpn) subunits, including PSMD8/Rpn12. One important function of the lid is to recognize multiubiquitinated proteins and other potential substrates of the proteasome (Glickman et al., 1998). The base contains three non-ATPases subunits (Rpn1, Rpn2 and Rpn10, this last common to both base and lid) and six putative ATPases subunits (Rpt1-6), including PSMC4/TBP7. These interact with non-native conformations of nonubiquitinated target proteins (Glickman et al., 1998; Voges et al., 1999; Strickland et al., 2000) and one of them S6′/TBP1, was found to bind aggregated or monomeric α-synuclein (Ghee et al., 2000; Snyder et al., 2003). In the same context, it has been recently reported that PSMC4, also called regulatory proteasomal protein S6, specifically interacts with both wild type and mutant synphilin-1 (Duke et al., 2004), another presynaptic protein, associated with synaptic vesicles (Ribeiro et al., 2002). Synphilin-1 was found associated with α-synuclein and, similarly, it accumulates in the Lewy body (Wakabayashi et al., 2000). Thus, the decreased levels of PSMC4 found in the present invention, together with the progressive inhibition of the UPS by the slow but persistent aggregation of α-synuclein, may cause their accumulation in Lewy bodies (Jellinger, 2003; Liani et al., 2004). The observed increase in the expression of the glycosaminoclycan heparan-sulphate gene in PD samples, may possible contribute as well to fibrillation and aggregation of α-synuclein, as suggested (Cohlberg et al., 2002). Each of the six ATPases is essential and they account for the ATP-requirement for proteolysis and for association of the 20S and 19S complexes to form the 26S complex (Ghislain et al., 1993; Gordon et al., 1993). Therefore, the decline in the expression of PSMC4/TBP7 in brains of Parkinsonian patients may contribute to decreased levels of 26S proteasome complex, abnormal accumulation of ubiquitinated proteins and reduced rates of degradation of short-lived proteins such as cyclins, which in turn may induce cell defects (for review see (Coux et al., 1996; Voges et al., 1999)). Indeed, we observed a decline in cyclin G associated kinase gene. Accumulation of cyclins and cyclin-dependent kinases, has been reported in post-mitotic neurons undergoing apoptosis (Padmanabhan et al., 1999; Copani et al., 2001) and in the MPTP model of PD (Grunblatt et al., 2001). It has been suggested that this event may represent an attempt at cell cycle re-entry (Verdaguer et al., 2002). Our findings are in line with a previous report of decreased expression of the 20S proteasome a-subunits, but not β-subunits, decreased protein expression levels of some 19S subunits and functional deficits in the 26/20S proteasome activity in the SNpc of patients with sporadic PD (McNaught et al., 2003). In this study however, the exact nature of the different subunits affected was not established, as the antibodies employed in the western blot analyses recognize sequences that are common to the various proteasome subunits and because of possible protein dimerization, leading to overestimation of the molecular weight. Thus, the present invention provides a wider view, revealing gene changes in the expression of specific proteasome components.

Heat Shock Protein Chaperone HSC-70

Proteotoxic insults to cells or several stress conditions can induce up regulation of molecular chaperones aimed at protecting cells by assisting to correct folding of wild type and mutated proteins. One of such is the 70 kDa heat-shock cognate protein (Hsc70) (Zinsmaier and Bronk, 2001), a member of the heat-shock protein 70 (Hsp70) family. It is suggested that Hsc70 normally mediates cellular processes such as protein folding, refolding, assembly, disassembly and protein translocation via biological membranes. It has been shown that overexpression of Hsp70 reduces the amount of misfolded, aggregated α-synuclein species in vivo and in vitro (Klucken et al., 2004), prevents the loss of dopaminergic neurons in a model of PD in transgenic flies that express either the wild-type α-synuclein, as well as the mutant forms A30P and A53T (Auluck et al., 2002) and suppresses the degeneration associated with polyglutamine pathogenicity (Bonini, 2002). Recently, a functional polymorphism in the 5′ promoter region of HSP70-1 has been reported in 274 PD patients, which may increase susceptibility to PD (Wu et al., 2004). We have found significant decreased gene expression of HSP8, coding for HSC70/HSC54, both in the SNpc and SNr of five out of six parkinsonian patients, as confirmed by real-time quantitative PCR, whereas no significant alterations were observed between cerebellum from PD patients and controls, suggestive of tissue specificity. HSPA8 exists in two isoforms, the truncated one lacking 153 amino acids residues in the protein binding and variable domain. (Tsukahara et al., 2000) It is thought to function as an endogenous inhibitory regulator of Hsc70 by competing the co-chaperones (Tsukahara et al., 2000). At present, we can not differentiate which of these are more avidly affected.

In addition to the involvement of Hsc70 in vesicular and nuclear trafficking they may play a role in protein ubiquitination through recognition of unfolded or aberrant proteins and delivery to a cochaperone, E3 ligase enzyme CHIP (carboxyl-terminus of Hsc70 interacting protein) (Murata et al., 2003). CHIP can cooperate with Hsp90 and/or Hsp70/Hsc70 and ubiquitinate their attached misfolded substrates. Thus the Hsc70-CHIP pair represents an E3 ligase for specifically recognizing unfolded proteins presented by the chaperones. This molecular chaperone-UPS quality control system is of extreme importance in neurodegenerative diseases, since proteins that evade refolding or degradation by the UPS form aggregates that accumulates into inclusion bodies. Indeed, molecular chaperones as well as ubiquitin and proteasome are recruited to inclusion bodies and Lewy Body (Stenoien et al., 1999; Sherman and Goldberg, 2001) indicating the attempt of the quality control system to degrade damaged proteins or prevent their removal. In fact, a number of neurodegenerative diseases appear to result from failure of the protein quality control system (Sherman and Goldberg, 2001).

Cell Adhesion Molecules, Iron and Oxidative Stress

An association between genes playing essential roles in neuronal development, such as cell migration and axonal elongation as well as components of the cytoskeleton and AD, has recently been suggested (De Ferrari and Inestrosa, 2000). A similar hypothesis can be assigned to PD where abnormal phosphorylation of cytoskeleton components (e.g. neurofilaments, microtubule-associated proteins) and ubiquitination of synaptic and other proteins, are encountered within the Lewy bodies (Jellinger, 2003).

Our present findings showing abnormal upregulation of genes with structural and phosphorylative molecular functions integral to membrane and extracellular matrix (cell adhesion process), support this assumption and extend our knowledge revealing potential new gene targets for future therapeutics.

During brain inflammation such as those reported in PD, AD and multiple sclerosis, the breakdown in the organization of key cell adhesion molecules and reduction in their signal transduction pathways may generate brain pathology, probably related to the recruitment of glia cells and macrophages and to the elevation in cytokines and OS. In addition, in five out of the six patients we observed a striking induction by more than 1.5 fold of EGLN 1 (egl nine homolog 1) gene, a recently described proline hydroxylase enzyme belonging to the iron- and 2-oxoglutarate-dependent dioxygenase superfamily (Epstein et al., 2001). These enzymes act as key iron and oxygen sensor controlling the expression of the transcription factor hypoxia-inducible factor-1 alpha (HIF), a master regulator orchestrating the coordinated induction of an array of hypoxia-sensitive genes. The target genes of HIF are especially related to angiogenesis, cell proliferation/survival and glucose/iron metabolism (Lee et al., 2004). Upon high oxygen levels or iron overload, the EGLN hydroxylases targets HIF to proteasomal degradation.

Interestingly, the free iron-induced proteasomal-mediated degradation of iron regulatory protein (IRP2) involves also activation of 2-oxoglutarate-dependent dioxygenases and is inhibited by iron chelators (Hanson et al., 2003; Wang et al., 2004). Thus, it is possible that IRP2 is a substrate of EGLN1, which causes post-translational modification, signaling it for protein degradation. Excessive production of EGLN1 in the SNpc may lead to a fall in IRP2 and subsequent decrease in transferrin receptor (TfR) MRNA and increase in ferritin levels, both subjected to positive and negative transcriptional regulation by IRP2, respectively (Meyron-Holtz et al., 2004; Ponka, 2004) Recent studies in knock-out mice for IRP2, have revealed accumulation of iron in the striatum with substantial bradykinesia and tremor (LaVaute et al., 2001).

Increased expression of the iron and OS sensor protein may be directly responsible for the observed reduction in phosphofructokinase and also the angiogenic factor VEGF, both regulated by the HIF proteins (Minchenko et al., 2003). Phosphofructokinase is the key regulatory enzyme that controls the glucose flux through the glycolytic pathway. Similarly, VEGF activates genes involved in glucose transport and metabolism via activation of the PI3K and ras pathways. The expression of two major players in these pathways, ras homolog gene family, member B and phosphoinositide-3-kinase, catalytic, alpha polypeptide, were also down-regulated in parkinsonian SNpc. This finding support previous reports in human PD patients using positron emission tomography (PET) analysis, demonstrating a decrease in glucose uptake into the SN (Berding et al., 2001) and give a wider view of major survival pathways affected by the disease. These observations, together with the additional decrease in a number of energy pathways/glycolysis-related genes, as well as increases in iron/OS and inflammatory genes, is consistent with the hypothesis that mitochondrial dysfunction and reactive oxygen and nitrogen species contribute to the pathogenesis of PD. In line with this, a recent study linked a recessive mutation in the putative free radical sensor DJ-1 gene, with early-onset parkinsonism (Bonifati et al., 2003).

To our surprise, apoptosis-related gene constituted the smallest functional class in PD samples, questioning the relevance of program cell death in the neurodegenerative cascade of events occurring in the disease. The role of apoptosis in PD is highly controversial since the evidence for it does not correlate with the pathological findings and the rate of neurodegeneration. We cannot exclude the possibility that apoptotic changes occurred at earlier stages and they decline with disease progression.

Dopamine Neurotransmission and Metabolism

The disruption in these neuronal processes may be exacerbated by the observed reduction in the expression of cell signaling genes, which may be coupled to cell adhesion protein complexes and in genes belonging to the vesicle secretory pathway and dopaminergic neurotransmission and metabolism in the parkinsonian SN. In a highly stringent analysis, sought to detect crucial alterations in at least five out of the six PD patients, with 1.5 fold change in gene expression relative to the control group, we observed major decreases in the expression of 8 genes, 3 of them related to DA transmission, including the vesicular monoamine transporter VMAT2 (SCL18A2). The real-time PCR confirmatory analysis revealed a specific reduction of VMAT2 mRNA in the SNpc while the changes in the SNr were less pronounced. This finding is in agreement with previous reports on post mortem SN of controls and PD showing a marked reduction of VMAT2 mRNA in PD which was associated with a marked reduction in both dopamine transporter and VMAT2 signal per cell in the remaining pigmented neurons (Harrington et al., 1996; Brooks, 2003). These alterations were specific for the SN as no significant difference was seen in the cerebellum between the PD and control samples. Two other striking changes were related to the reduction in DA transmission and metabolism-related genes ALDH1A1 and ARPP-21 coding for aldehyde dehydrogenase (ALDH) and cAMP regulated phosphoprotein, respectively. ARPP-21 is specifically enriched in DA-innervated brain regions of the basal ganglia (e.g. caudate-putamen), and in the substantia nigra (Ouimet et al., 1989; Tsou et al., 1993). Similar to DARPP-32 (dopamine and adenosine 3′:5′-monophosphate-regulated phosphoprotein-32K), ARPP-21 is activated by DA receptor D1, and thus may represent an index of functional activity of D-1 neurotransmission. As far as we know, this is the first report of decreased ARPP-21 mRNA levels in PD SN. ALDH1A1 was found to be expressed highly and specifically in DA cells of the SN and ventral tegmental area (VTA) and to be markedly reduced in SNpc dopaminergic neurons but not in those of the VTA of PD brains (Galter et al., 2003). This is in line with observation that striatal ALDH activity in 6-hydroxydopamine or electrical induced lesion in rats (Agid et al., 1973) or cats (Duncan et al., 1972), was significantly reduced.

ALDHs are involved in the degradation of DA to aldehyde derivatives (3,4dihydroxyphenylacetaldehyde and 4-hydroxy-3-methoxyphenylacetaldehyde) (Mardh and Vallee, 1986), which are then metabolized to homovanilic acid and dihydroxyphenylacetic acid and in detoxification of aldehydes which are highly reactive and neurotoxic (Hjelle and Petersen, 1983). Thus, alteration in DA transmission may alter ALDH activity and/or, conversely, changes in ALDH-mediated metabolism may affect DA levels in nerve cell bodies and terminal fields in basal ganglia and the limbic system. These proteins, in conjunction with VMAT2 may now be considered new markers for PD.

The overall decrease in the levels of these DA neurotransmission and metabolism-related genes observed in the present invention, may seriously compromise neurotransmitter storage and correlate with DA neuron dysfunction. This may further be exacerbated by the reduced levels of SEC22L1 mRNA, a member of the SEC22 family of early vesicle trafficking proteins from the endoplasmic reticulum to (Hay et al., 1996), and by the observed reduction in many membrane carrier transporters. Impairment of cell transport and vesicle trafficking, carrying a wide repertoire of intracellular and membrane proteins, may well account for their aggregation and cell deposition into cytoplasmic inclusions (aggresomes) and in the Lewy body. One simple hypothesis explaining our observations is that loss of DA-containing neuronal bodies and neuronal synapses may account for the reduce levels of the gene products. This assumption would predict similar gene expression changes in a vast repertoire of vesicle traffic or synaptic-related genes. However, as emerged from our microarray and real-time PCR confirmations, the expression of many genes related to these categories such as Rab3b, syntaxin 6 and COP zeta 2, was not altered in PD.

Protein Modification/Phosphorylation

The expression of two pre-mRNA splicing related genes was inversely and highly affected in each of the five or six PD samples compared to control brains: the SRPK2 gene coding for the SFRS protein kinase 2, was down-regulated and SRRM2 coding for serine/arginine repetitive matrix 2, was significantly increased. SRPK2 is a kinase highly expressed in brain with a predicted sequence for specific phosphorylation of arginine/serine-rich (SR) domain-containing splicing factors, which in turn regulate RNA splicing in brain regions (Wang et al., 1998). Interestingly, SRRM2 (or SRm300), is a SR domain-containing splicing factor functioning as a co-activator within a major complex SRm 160/300, responsible for the processing of a subset of constitutively spliced pre-mRNAs (Blencowe et al., 2000).

If SRMM2 is a substrate of SRPK2, then it seems plausible that the robust decline in the kinase SRPK2 and the high expression of SRMM2 in PD SNpc, leads to abnormal hypophosphorylated SRMM2 accumulation. This in turn may affect the nuclear distribution of SRm160 and/or other associated SR proteins converging eventually in a broad impairment in splicing activity.

Conclusion

In spite of the vast information regarding the pathology and etiology of PD, it is still premature to assert what is the primary event(s) that triggers the development of PD. The present invention shows a reduction in the levels of selective components of the UPS together with a progressive misregulation of extracellular matrix/cytoskeleton components, concurrent with a state of OS and inflammation. These series of events may act independently or cooperatively during the course of the disease, leading eventually to the demise of dopaminergic neurons. Thus, subtle alterations in the kinetics of the different affected proteins may have, during the decades, a cumulative effect underlying the slowly progressive neurodegeneration of the DA-containing neurons in PD.

TABLE 1
Summary of case selection of SNpc, SNr and cerebellum
regions for the Gene Chip array and/or quantitative
real-time RT-PCR analysis.

| | Braak & | | | | |
|---|---|---|---|---|---|
| | Braak/ | | | | |
| Case | Hoehn & | | | | |
| Number | Yahr | Age | Sex | PMD (Hr) | Cause of death |
| P1 | H&Y 3/4 | 73 | F | 19 | Parkinson/renal |
| | | | | | insufficiency |
| P2 | H&Y 5/6 | 79 | F | 17 | Parkinson/heart |
| | | | | | failure |
| P3 | H&Y 3/4 | 87 | F | 48 | n.a. |
| P4 | H&Y 3/4 | 75 | M | 24 | Parkinson/ |
| | | | | | Pneumonia |
| P5 | H&Y 5; | 78 | F | 29 | Parkinson/heart |
| | B&B II | | | | failure |
| P6 | H&Y 3 | 70 | M | 22 | Global heart |
| | | | | | failure |
| P7 | H&Y 5 | 65 | M | 68 | Parkinson/Global |
| | | | | | heart failure |
| C1 | | 72 | M | 23 | ventricular |
| | | | | | fibrillation |
| C2 | | 86 | F | 20 | renal insufficiency |
| C3 | | 75 | M | 21 | Toxic heart failure |
| C4 | | 85 | M | 20.5 | Left side heart |
| | | | | | failure |
| C5 | | 88 | M | 48 | acute renal failure |
| C6 | | 68 | M | 24 | n.a. |
| C7 | | 85 | M | 25 | heart failure |

Post-Mortem delay, PMD; Not available, n.a.

TABLE 2

Sequences of oligonucleotide primer pairs and probes
labelled with FAM used for real-time quantitative PCR

| | | | |
|---|---|---|---|
| Gene (GeneBank | Primer pairs | Primer pairs | Product |

| | | | | | |
|---|---|---|---|---|---|
| accession | sequence | sequence | size | Cycle | Reaction |

| | | | | | |
|---|---|---|---|---|---|
| number) | (5′→3′) | (5′→3′) | (bp) | No. | Efficiency |

| | | | | |
|---|---|---|---|---|
| 18s ribosomal | n.a. (QuantiTect | 150 | 36 | 95 |
| (V01270) | Hs_RRN18S Assay) | | | |
| ACTB | n.a. (QuantiTect | 150 | 40 | 87.9 |
| (NM_001101) | Hs_ACTB Assay) | | | |
| ALAS1 | n.a. (QuantiTect | 100 | 40 | 80.7 |
| (NM_000688) | Hs_ALAS1 Assay) | | | |
| GAPDH | n.a. (QuantiTect | 130 | 35 | 89 |
| (NM_002046) | Hs_GAPD Assay) | | | |
| RPL13A | n.a. (QuantiTect | 100 | 35 | 99.5 |
| (NM_012423) | Hs_RPL13A Assay) | | | |

| | | | | | |
|---|---|---|---|---|---|
| VMAT2 | CTG TAT GTT | TTC CAG TCT | 119 | 45 | 88.3 |
| (AI269290) | CTT TGT TCT | TGC TAA CC | | | |
| | GGT AGA T*A | SEQ ID NO: 2 | | | |
| | GCT *T* GT* | | | | |
| | ACT TGG G | | | | |
| | SEQ ID NO: 1 | | | | |
| HSP 70 kDa | TGC AGT TGG | CTC TTG TGT | 107 | 36 | 97.5 |
| (AB034951.1) | TAT TGA TCT | GG* GTG TT | | | |
| | TGG TAG CTT | SEQ ID NO: 4 | | | |
| | GGA GTG GTT | | | | |
| | CGG TT | | | | |
| | SEQ ID NO: 3 | | | | |
| RAB3B | GGGACA ATG | TGG AGG AAG | 107 | 40 | 99.6 |
| (BC005035.1) | CAC AAG TTA | AGA GG* GT | | | |
| | T AAT CAA | SEQ ID NO: 6 | | | |
| | ACC CAA GCT | | | | |
| | GCT CT | | | | |
| | SEQ ID NO: 5 | | | | |
| SKP1A | CAG CAG GGC | GG* GAG GCA | 99 | 40 | 99.5 |
| (NM_006930.1) | AGA ATA AAA | AAG AAA GGA | | | |
| | AC GAGA AGA | A | | | |
| | ACA GAT GGA | SEQ ID NO: 8 | | | |
| | AA | | | | |
| | SEQ ID NO: 7 | | | | |
| Syntaxin6 | GTG GTG AAA | CAC AGC AAC | 118 | 36 | 99.9 |
| (NM_005819.1) | GGA GAG GTA | AAG G*G AAG | | | |
| | TTG GTG GTC | SEQ ID NO: 10 | | | |
| | CAG TCG ATT | | | | |
| | SEQ ID NO: 9 | | | | |
| COP zeta 2 | TTG ACT GAA | TGA AAT GAA | 118 | 40 | 98.7 |
| (NM_016429.1) | CAG AGT GTG | GGC TGT GG | | | |
| | G GAT TGG | SEQ ID NO: 12 | | | |
| | *GG AAA TGG | | | | |
| | TCT GG | | | | |
| | SEQ ID NO: 11 | | | | |

*Indicates modified nucleotide
n. a. not Available

TABLE 3
Down regulated genes in SNpc of Pakinson's Disease

| | | | FOLD OF |
|---|---|---|---|
| GENE BANK | SYMBOL | GENENAME | CONTROL |
| | | *Proteolysis and Peptidolysis | |
| NM_006503.1 | PSMC4 | proteasome (prosome, macropain) 26S subunit, | 0.52 |
| | | ATPase, 4 | |
| NM_002790.1 | PSMA5 | proteasome (prosome, macropain) subunit, | 0.47 |
| | | alpha type, 5 | |
| NM_002787.1 | PSMA2 | proteasome (prosome, macropain) subunit, | 0.65 |
| | | alpha type, 2 | |
| NM_002788.1 | PSMA3 | proteasome (prosome, macropain) subunit, | 0.42 |
| | | alpha type, 3 | |
| NM_005339.2 | HIP2 | huntingtin interacting protein 2 | 0.54 |
| NM_002570.1 | PACE4 | paired basic amino acid cleaving system 4 | 0.59 |
| NM_006930.1 | SKP1A | S-phase kinase-associated protein 1A (p19A) | 0.51 |
| | | Dopaminergic Transmission/Metabolism | |
| NM_000689.1 | ALDH1A1 | aldehyde dehydrogenase 1 family, member A1 | 0.35 |
| NM_016300.1 | ARPP-21 | cyclic AMP-regulated phosphoprotein, 21 kD | 0.51 |
| | | Energy Pathways/Glycolysis | |
| NM_004373.1 | COX6A1 | cytochrome c oxidase subunit VIa polypeptide 1 | 0.58 |
| NM_002627.1 | PFKP | phosphofructokinase, platelet | 0.66 |
| NM_000436.1 | OXCT | 3-oxoacid CoA transferase | 0.57 |
| NM_000158.1 | GBE1 | glucan (1,4-alpha-), branching enzyme 1 | 0.45 |
| | | (glycogen branching enzyme, Andersen disease, | |
| | | glycogen storage disease type IV) | |
| AV727381 | UQCRC2 | ubiquinol-cytochrome c reductase core protein | 0.59 |
| | | II | |
| | | Signal Transduction | |
| NM_006055.1 | LANCL1 | LanC lantibiotic synthetase component C-like 1 | 0.57 |
| | | (bacterial) | |
| NM_004236.1 | TRIP15 | thyroid receptor interacting protein 15 | 0.63 |
| NM_006218.1 | PIK3CA | phosphoinositide-3-kinase, catalytic, alpha | 0.5 |
| | | polypeptide | |
| NM_006226.1 | PLCL1 | phospholipase C-like 1 | 0.48 |
| NM_005274.1 | GNG5 | guanine nucleotide binding protein (G protein), | 0.66 |
| | | gamma 5 | |
| AL049933.1 | GNAI1 | guanine nucleotide binding protein (G protein), | 0.64 |
| | | alpha inhibiting activity polypeptide 1 | |
| AF022375.1 | VEGF | vascular endothelial growth factor | 0.49 |
| AI263909 | RHOB | ras homolog gene family, member B | 0.61 |
| S77154.1 | NR4A2 | nuclear receptor subfamily 4, group A, member | 0.6 |
| | | 2 | |
| | | Transport | |
| NM_001860.1 | SLC31A2 | solute carrier family 31 (copper transporters), | 0.62 |
| | | member 2 | |
| NM_004731.1 | SLC16A7 | solute carrier family 16 (monocarboxylic acid | 0.46 |
| | | transporters), member 7 | |
| BC004443.1 | ATP6V1E1 | ATPase, H+ transporting, lysosomal 31 kDa, V1 | 0.59 |
| | | subunit E isoform 1 | |
| NM_005827.1 | UGTREL1 | solute carrier family 35, member B1 SEC22 | 0.42 |
| | | vesicle trafficking | |
| AA890010 | SEC22L1 | protein-like 1 (S. cerevisiae) | 0.55 |
| AI269290 | SLC18A2 | solute carrier family 18 (vesicular monoamine), | |
| | | member 2 | |
| | | Cell Adhesion | |
| NM_001769.1 | CD9 | CD9 antigen (p24) | 0.39 |
| NM_021153.1 | CDH19 | cadherin 19, type 2 | 0.5 |
| | | Cell Cycle | |
| NM_004417.2 | DUSP1 | dual specificity phosphatase 1 | 0.57 |
| BC004421.1 | HSA6591 | zinc finger protein 330 | 0.63 |
| | | Cell Motility/Cytoskeleton | |
| NM_005721.2 | ACTR3 | ARP3 actin-related protein 3 homolog (yeast) | 0.44 |
| NM_004520.1 | KIF2 | kinesin heavy chain member 2 | 0.52 |
| BC004188.1 | TUBB2 | tubulin, beta, 2 | 0.62 |
| | | Lipid Biosynthesis | |
| NM_002979.1 | SCP2 | sterol carrier protein 2 | 0.58 |
| BC004100.1 | PIGH | phosphatidylinositol glycan, class H | 0.44 |
| | | Nucleobase, Nucleoside, Nucleotide and | |
| | | Nucleic Acid Metabolism | |
| BC000422.1 | ARIH2 | ariadne homolog 2 (Drosophila) | 0.61 |
| NM_016576.1 | GMPR2 | guanosine monophosphate reductase 2 | 0.47 |
| | | Phosphate Metabolism | |
| NM_021129.1 | PP | pyrophosphatase (inorganic) | 0.56 |
| | | Protein Modification/Phosphorylation | |
| NM_003640.1 | IKBKAP | inhibitor of kappa light polypeptide gene | 0.65 |
| | | enhancer in B-cells, kinase complexassociated | |
| | | protein | |
| NM_002731.1 | PRKACB | protein kinase, cAMPdependent, catalytic, beta | 0.57 |
| NM_002847.1 | PTPRN2 | protein tyrosine phosphatase, receptor type, N | 0.55 |
| | | polypeptide 2 | |
| NM_003138.1 | SRPK2 | SFRS protein kinase 2 | 0.46 |
| | | RNA Processing | |
| NM_005872.1 | BCAS2 | breast carcinoma amplified sequence 2 | 0.58 |
| NM_013417.1 | IARS | isoleucine-tRNA synthetase | 0.36 |
| NM_002713.1 | PPP1R8 | protein phosphatase 1, regulatory (inhibitor) | 0.51 |
| | | subunit 8 | |
| | | Others and Unknown Function | |
| NM_004261.1 | SEP15 | 15 kDa selenoprotein | 0.62 |
| NM_003187.1 | TAF9 | TAF9 RNA polymerase II, TATA box binding | 0.66 |
| | | protein (TBP)-associated factor, 32 kDa | |
| NM_005667.1 | ZFP103 | neuroendocrine differentiation factor | 0.62 |
| NM_004627.1 | WRB | tryptophan rich basic protein | 0.6 |
| NM_014255.1 | TMEM4 | transmembrane protein 4 | 0.6 |
| AI760760 | SMARCA3 | SWI/SNF related, matrix associated, actin | 0.66 |
| | | dependent regulator of chromatin, subfamily a, | |
| | | member 3 | |
| AI743037 | FMR1 | neuroendocrine differentiation factor | 0.65 |
| NM_002601.1 | PDE6D | phosphodiesterase 6D, cGMPspecific, rod, delta | 0.64 |
| NM_003919.1 | SGCE | sarcoglycan, epsilon | 0.63 |
| NM_001698.1 | AUH | AU RNA binding protein/enoyl- Coenzyme A | 0.6 |
| | | hydratase | |
| BF439316 | TMEFF1 | transmembrane protein with EGF-like and two | 0.54 |
| | | follistatin-like domains 1 | |
| NM_000049.1 | ASPA | aspartoacylase (aminoacylase 2, Canavan | 0.56 |
| | | disease) | |
| AL136939.1 | HELO1 | ELOVL family member 5, elongation of long | 0.6 |
| | | chain fatty acids (FEN1/Elo2, SUR4/Elo3- like, | |
| | | yeast) | |
| M30471.1 | ADH5 | alcohol dehydrogenase 5 class III), chi | 0.62 |
| | | polypeptide | |
| AF161522.1 | C3orf4 | chromosome 3 open reading frame 4 | 0.47 |
| BC002511.1 | CBR1 | carbonyl reductase 1 | 0.51 |
| AI984005 | XPOT | exportin, tRNA (nuclear export receptor for | 0.49 |
| | | tRNAs) | |
| NM_016139.1 | LOC51142 | phosphorylase kinase, gamma 1 (muscle) | 0.66 |
| NM_016121.1 | NY-REN-45 | potassium channel tetramerisation domain | 0.5 |
| | | containing 3 | |
| NM_022118.1 | SE70-2 | chromosome 13 open reading frame 10 | 0.66 |
| NM_018700.1 | TRIM36 | tripartite motif-containing 36 | 0.42 |
| AB034951.1 | HSPA8 | heat shock 70 kDa protein 8 | |

Genes are clustered into groups by biological function. The mean fold-change of each gene in PD samples relative to control samples as well as the gene symbol and GenBank accession number are indicated.
*specific categories determined to be significantly over-represented using the statistical clustering program EASE, are indicated

TABLE 4
Up regulated genes in SNpc of Pakinson's Disease

| | | | FOLD |
|---|---|---|---|
| GENBANK | SYMBOL | GENE | CHANGE |
| | | *Cell Adhesion | |
| NM_014288.1 | ITGB3BP | integrin beta 3 binding protein beta3- | 1.67 |
| | | endonexin) | |
| NM_000632.2 | ITGAM | integrin, alpha M (complement component | 1.54 |
| | | receptor 3, alpha; also known as CD11b | |
| | | (p170), macrophage antigen alpha | |
| | | polypeptide) | |
| AF018081.1 | COL18A1 | collagen, type XVIII, alpha 1 | 1.57 |
| AI741056 | SELPLG | selectin P ligand | 2.10 |
| AF065389.1 | TM4SF9 | transmembrane 4 superfamily member 9 | 1.60 |
| AK022316.1 | PARVA | parvin, alpha | 1.80 |
| X79683.1 | LAMB2 | laminin, beta 2 (laminin S) | 1.76 |
| NM_006043.1 | HS3ST2 | heparan sulfate (glucosamine) 3-O- | 1.84 |
| | | sulfotransferase 2 | |
| U58766 | TSTA3 | tissue specific transplantation antigen P35B | 1.60 |
| AI984221 | COL5A3 | collagen, type V, alpha 3 | 1.68 |
| | | Cell Motility/Cytoskeleton | |
| NM_002579.1 | PALM | paralemmin | 1.59 |
| NM_003803.1 | MYOM1 | myomesin 1 (skelemin) 185 kDa | 2.13 |
| M62994.1 | FLNB | filamin B, beta (actin binding protein 278) | 1.75 |
| | | Development | |
| NM_000190.1 | HMBS | hydroxymethylbilane synthase | 1.65 |
| NM_000423.1 | KRT2A | keratin 2A (epidermal ichthyosis bullosa of | 2.04 |
| | | Siemens) | |
| NM_020163.1 | LOC56920 | semaphorin sem2 | 1.98 |
| | | Cell Cycle | |
| NM_004383.1 | CSK | c-src tyrosine kinase | 1.59 |
| AF241788.1 | NUDC | nuclear distribution gene C homolog (A. | 1.52 |
| | | nidulans) | |
| NM_007076.1 | HYPE | Huntingtin interacting protein E | 1.54 |
| D88435 | GAK | cyclin G associated kinase | 1.54 |
| | | Immune Response | |
| AI743792 | SIAT1 | sialyltransferase 1 (betagalactoside alpha-2,6- | 1.79 |
| | | sialyltransferase) colony stimulating factor 1 | |
| NM_005211.1 | CSF1R | receptor, formerly McDonough feline | 1.57 |
| | | sarcoma viral (v-fms) oncogene homolog | |
| AI073984 | ICSBP1 | interferon consensus sequence binding | 1.88 |
| | | protein 1 | |
| X59350 | CD22 | CD22 antigen | 1.60 |
| | | Response to Stress | |
| NM_001983.1 | ERCC1 | excision repair crosscomplementing rodent | 1.61 |
| | | repair deficiency, complementation group 1 | |
| | | (includes overlapping antisense sequence) | |
| AK023253.1 | DNAJB5 | DnaJ (Hsp40) homolog, subfamily B, | 1.73 |
| | | member 5 | |
| | | Apoptosis | |
| AI721219 | TRAF3 | TNF receptor-associated factor 3 | 1.63 |
| | | Nucleobase, Nucleoside, Nucleotide and | |
| | | Nucleic Acid Metabolism | |
| NM_004349.1 | CBFA2T1 | core-binding factor, runt domain, alpha | 1.59 |
| | | subunit 2; translocated to, 1; cyclin D-related | |
| N25915 | CUGBP1 | CUG triplet repeat, RNA binding protein 1 | 1.52 |
| AA887083 | ING4 | inhibitor of growth family, member 4 | 1.52 |
| | | Transcription | |
| BC004973.1 | STAT6 | signal transducer and activator of | 1.82 |
| | | transcription 6, interleukin-4 induced | |
| NM_005674.1 | ZNF239 | zinc finger protein 239 | 1.56 |
| NM_003189.1 | TAL1 | T-cell acute lymphocytic leukemia 1 | 1.67 |
| AF118094.1 | TAF11 | TAF11 RNA polymerase II, TATA box | 1.59 |
| | | binding protein (TBP)- associated factor, | |
| | | 28 kDa | |
| AK024501.1 | MXD4 | MAX dimerization protein 4 | 1.82 |
| | | Protein Biosynthesis | |
| NM_004994.1 | MMP9 | matrix metalloproteinase 9 (gelatinase B, | 2.09 |
| | | 92 kDa gelatinase, 92 kDa type IV | |
| | | collagenase) | |
| BE966878 | EIF4G1 | eukaryotic translation initiation factor 4 | 1.69 |
| | | gamma, 1 | |
| BC005057.1 | EIF4EBP2 | eukaryotic translation initiation factor 4E | 1.75 |
| | | binding protein 2 | |
| NM_015414.1 | RPL36 | ribosomal protein L36 | 1.67 |
| BC005369.1 | EGLN1 | egl nine homolog 1 (C. elegans) | 2.37 |
| | | Protein Modification/Phosphorylation | |
| AW082913 | SRPK1 | SFRS protein kinase 1 | 1.53 |
| AL530441 | CSNK1G2 | casein kinase 1, gamma 2 | 1.58 |
| NM_002953.1 | RPS6KA1 | ribosomal protein S6 kinase, 90 kDa, | 1.67 |
| | | polypeptide 1 | |
| U68567.1 | MAN2B1 | mannosidase, alpha, class 2B, member 1 | 2.09 |
| AF181985.1 | JIK | STE20-like kinase | 1.66 |
| | | Signal Transduction | |
| NM_005475.1 | LNK | lymphocyte adaptor protein | 1.51 |
| NM_005541.1 | INPP5D | inositol polyphosphate-5-phosphatase, | 1.53 |
| | | 145 kDa | |
| NM_006211.1 | PENK | proenkephalin | 2.22 |
| | | Transport | |
| AW167713 | TCOF1 | Treacher Collins-Franceschetti syndrome 1 | 1.71 |
| BC001889.1 | NAPG | N-ethylmaleimide-sensitive factor attachment | 1.62 |
| | | protein, gamma | |
| BC003068.1 | SLC19A1 | solute carrier family 19 (folate transporter), | 1.59 |
| | | member 1 | |
| AF064243 | ITSN1 | intersectin 1 (SH3 domain protein) | 1.56 |
| | | Other/Unknown | |
| NM_015853.1 | LOC51035 | ORF | 1.57 |
| NM_006556.1 | PMVK | phosphomevalonate kinase | 1.54 |
| NM_004928.1 | C21orf2 | chromosome 21 open reading frame 2 | 1.83 |
| NM_016938.1 | EFEMP2 | EGF-containing fibulin-like extracellular | 1.93 |
| | | matrix protein 2 | |
| AI655799 | SRRM2 | serine/arginine repetitive matrix 2 | 1.82 |
| AV753028 | TBL1X | transducin (beta)-like 1×-linked | 1.65 |
| AA927724 | APRT | adenine phosphoribosyltransferase | 1.51 |
| AA679297 | SPUF | secreted protein of unknown function | 1.70 |
| NM_015710.1 | GLTSCR2 | glioma tumor suppressor candidate region | 1.60 |
| | | gene 2 | |
| NM_022371.1 | ADIR | torsin family 3, member A | 1.52 |
| NM_015926.1 | ZSIG11 | putative secreted protein ZSIG11 | 1.79 |
| NM_013385.2 | PSCD4 | pleckstrin homology, Sec7 and coiled-coil | 2.05 |
| | | domains 4 | |
| NM_005771.1 | RDHL | dehydrogenase/reductase (SDR family) | 1.81 |
| | | member 9 | |
| NM_016202.1 | LOC51157 | zinc finger protein 580 | 1.87 |
| AF074264 | LRP6 | low density lipoprotein receptor-related | 2.10 |
| | | protein 6 | |
| AC005943 | MBD3 | methyl-CpG binding domain protein 3 | 1.66 |
| AL042496 | C9orf7 | chromosome 9 open reading frame 7 | 1.70 |
| NM_009587.1 | LGALS9 | Binds galactosides. may play a role in | |
| | | thymocyte- epithelial interactions relevant | |
| | | to the biology of the thymus. The short | |
| | | isoform acts as an eosinophil | |
| | | chemoattractant. | |
| NM_021972.1 | SPHK1 | Catalyzes the phosphorylation of | |
| | | sphingosine to form sphingosine 1- | |
| | | phosphate (SPP), a lipid mediator with | |
| | | both intra- and extracellular functions. | |
| | | Also acts on D-erythro-sphingosine and | |
| | | to a lesser extent sphinganine, but not | |
| | | other lipids, such as D,L-threo- | |
| | | dihydrosphingosine, N,N- | |
| | | dimethylsphingosine, diacylglycerol, | |
| | | ceramide, or phosphatidylinositol. | |

*specific categories determined to be significantly over-represented using the statistical clustering program EASE, are indicated

### REFERENCES

Agid Y, Javoy F and Youdim M B H (1973) Monoamine oxidase and aldehyde dehydrogenase activity in the striatum of rats after 6-hydroxydopamine lesion of the nigrostriatal pathway. Br J Pharmacol 48(1): 175-178.

Auluck P K, Chan H Y, Trojanowski J Q, Lee V M and Bonini N M (2002) Chaperone suppression of alpha-synuclein toxicity in a Drosophila model for Parkinson's disease. Science 295(5556): 865-868.

Azzouz M, Martin-Rendon E, Barber R D, Mitrophanous K A, Carter E E, Rohll J B, Kingsman S M, Kingsman A J, Mazarakis N D. (2002) Multicistronic lentiviral vector-mediated striatal gene transfer of aromatic L-amino acid decarboxylase, tyrosine hydroxylase, and GTP cyclohydrolase I induces sustained transgene expression, dopamine production, and functional improvement in a rat model of Parkinson's disease. J Neurosci.; 22(23):10302-12.

Berding G, Odin P, Brooks D J, Nikkhah G, Matthies C, Peschel T, Shing M, Kolbe H, van Den Hoff J, Fricke H, Dengler R, Samii M and Knapp W H (2001) Resting regional cerebral glucose metabolism in advanced Parkinson's disease studied in the off and on conditions with [(18)F]FDG-PET. Mov Disord 16(6): 1014-1022.

Bernheimer H, Birkmayer W, Homykiewicz O, Jellinger K, Seitelberger F. (1973) Brain dopamine and the syndromes of Parkinson and Huntington. Clinical, morphological and neurochemical correlations. J Neurol Sci. 20(4):415-55.

Blencowe B J, Bauren G, Eldridge A G, Issner R, Nickerson J A, Rosonina E and Sharp P A (2000) The SRm160/300 splicing coactivator subunits. RNA 6(1): 111-120.

Bonifati V, Rizzu P, van Baren M J, Schaap O, Breedveld G J, Krieger E, Dekker M C, Squitieri F, Ibanez P, Joosse M, van Dongen J W, Vanacore N, van Swieten J C, Brice A, Meco G, van Duijn C M, Oostra B A and Heutink P (2003) Mutations in the DJ-1 gene associated with autosomal recessive early-onset parkinsonism. Science 299(5604): 256-259.

Bonini N M (2002) Chaperoning brain degeneration. PNAS 99(90004): 16407-16411.

Braak H and Braak E (1997) Diagnostic criteria for neuropathologic assessment of Alzheimer's disease. Neurobiology Aging 18(4 Suppl): S85-88.

Brooks D J. (1998) The early diagnosis of Parkinson's disease. Ann Neurol. 44(3 Suppl 1):S10-8.

Brooks D J (2003) Imaging end points for monitoring neuroprotection in Parkinson's disease. Ann Neurol 53 Suppl 3:S110-118; discussion SI 18-119.

Cadet J L, Jayanthi S, McCoy M T, Vawter M, Ladenheim B. (2001 ) Temporal profiling of methamphetamine-induced changes in gene expression in the mouse brain: evidence from cDNA array. Synapse. 41(1):40-8.

Ciechanover A and Brundin P (2003) The ubiquitin proteasome system in neurodegenerative diseases: sometimes the chicken, sometimes the egg. Neuron 40(2): 427-446.

Cohlberg J A, Li J, Uversky V N and Fink A L (2002) Heparin and other glycosaminoglycans stimulate the formation of amyloid fibrils from alpha-synuclein in vitro. Biochemistry 41(5): 1502-1511.

Copani A, Uberti D, Sortino M A, Bruno V, Nicoletti F and Memo M (2001) Activation of cell-cycleassociated proteins in neuronal death: a mandatory or dispensable path? Trends Neurosci 24(1): 25-31.

Coux O, Tanaka K and Goldberg A L (1996) Structure and functions of the 20S and 26S proteasomes. Annu Rev Biochem 65:801-847.

Dauer W and Przedborski S (2003) Parkinson's disease: mechanisms and models. Neuron 39(6): 889-909.

Dawson T M and Dawson V L (2003) Molecular pathways of neurodegeneration in Parkinson's disease. Science 302(5646): 819-822.

De Ferrari G V and Inestrosa N C (2000) Wnt signaling function in Alzheimer's disease. Brain Res Brain Res Rev 33(1): 1-12.

Dennis G. Jr., Sherman B T, Hosack D A, Yang J, Gao W, Lane H C and Lempicki R A (2003) DAVID: Database for Annotation, Visualization, and Integrated Discovery. Genome Biol 4(5): P3.

Dev K K, van der Putten H, Sommer B and Rovelli G (2003) Part I: parkin-associated proteins and Parkinson's disease. Neuropharmacology 45(1): 1-13.

Drukarch B, van Muiswinkel F L. (2001) Neuroprotection for Parkinson's disease: a new approach for a new millennium. Expert Opin Investig Drugs. 10(10):1855-68.

Duke D C, Moran F E, Turkheimer D T, Dexter R K, Pearce M B and Graeber M B (2004) Expression profiling of the parkinsonian substantia nigra using microarrays. Mov Disord 19(Suppl. 9): S38.

Duncan R J, Sourkes T L, Boucher R, Poirier L J and Roberge A (1972) Aldehyde dehydrogenase and monoamine oxidase in the striatum of cats with nigrostriatal lesions. J Neurochem 19(8): 2007-2010.

Drukarch B, van Muiswinkel F L. (2001) Neuroprotection for Parkinson's disease: a new approach for a new millennium. Expert Opin Investig Drugs. 10(10):1855-68.

Ebadi M, Govitrapong P, Sharma S, Muralikrishnan D, Shavali S, Pellett L, Schafer R, Albano C and Eken J (2001) Ubiquinone (coenzyme q10) and mitochondria in oxidative stress of parkinson's disease. Biol Signals Recept 10(3-4): 224-253.

Epstein A C, Gleadle J M, McNeill L A, Hewitson K S, O'Rourke J, Mole D R, Mukherji M, Metzen E, Wilson M I, Dhanda A, Tian Y M, Masson N, Hamilton D L, Jaakkola P, Barstead R, Hodgkin J, Maxwell P H, Pugh C W, Schofield C J and Ratcliffe P J (2001) C. elegans EGL-9 and mammalian homologs define a family of dioxygenases that regulate HIF by prolyl hydroxylation. Cell 107(1): 43-54.

Fahn, S., Elton, R. and committee, m.o.t.U.d., Unified Parkinson's disease rating scale. In S. Fahn, C. Mardsen and M. Goldstein (Eds.), Recent development in a Parkinson's disease, Macmillan, New York, 1987, pp. 153-167.

Felletschin B, Bauer P, Walter U, Behnke S, Spiegel J, Csoti I, Sommer U, Zeiler B, Becker G, Riess O and Berg D (2003) Screening for mutations of the ferritin light and heavy genes in Parkinson's disease patients with hyperechogenicity of the substantia nigra. Neurosci Lett 352(1): 53-56.

Futatsumori M, Kasai K, Takatsu H, Shin H W and Nakayama K (2000) Identification and characterization of novel isoforms of COP I subunits. J Biochem 128(5): 793-801.

Galter D, Buervenich S, Carmine A, Anvret M and Olson L (2003) ALDH1 mRNA: presence in human dopamine neurons and decreases in substantia nigra in Parkinson's disease and in the ventral tegmental area in schizophrenia. Neurobiol Dis 14(3): 637-647.

Ghee M, Fournier A and Mallet J (2000) Rat alpha-Synuclein Interacts with Tat Binding Protein 1, a Component of the 26S Proteasomal Complex. J Neurochem 75(5): 2221-2224.

Ghislain M, Udvardy A and Mann C (1993) S. cerevisiae 26S protease mutants arrest cell division in G2/metaphase. Nature 366(6453): 358-362.

Glickman M H and Ciechanover A (2002) The ubiquitin-proteasome proteolytic pathway: destruction for the sake of construction. Physiology Review 82(2): 373-428.

Glickman M H, Rubin D M, Coux O, Wefes I, Pfeifer G, Cjeka Z, Baumeister W, Fried V A and Finley D (1998) A subcomplex of the proteasome regulatory particle required for ubiquitin-conjugate degradation and related to the COP9-signalosome and eIF3. Cell 94(5): 615-623.

Gonzalez L, Jr. and Scheller R H (1999) Regulation of membrane trafficking: structural insights from a Rab/effector complex. Cell 96(6): 755-758.

Gordon C, McGurk G, Dillon P, Rosen C and Hastie N D (1993) Defective mitosis due to a mutation in the gene for a fission yeast 26S protease subunit. Nature 366(6453): 355-357.

Gotz M E, Kunig G, Riederer P and Youdim M B (1994) Oxidative stress: free radical production in neural degeneration. Pharmacology & Therapeutics 63(1): 37-122.

Grunblatt E, Mandel S, Maor G and Youdim M B H (2001) Gene expression analysis in N-methyl-4-phenyl-1,2,3,6-tetrahydropyridine mice model of Parkinson's disease using cDNA microarray:effect of R-apomorphine. J Neurochem 78(1):1-12.

Grunblatt E, Mandel S, Maor G, Youdim M B. (2001) Effects of R- and S-apomorphine on MPTP-induced nigro-striatal dopamine neuronal loss. J Neurochem. 2001 April; 77(1):146-56.

Grunblatt E, Schlosser R, Gerlach M, Riederer P. (2003) Preclinical versus clinical neuroprotection. Adv Neurol. 91:309-28.

Hanson E S, Rawlins M L and Leibold E A (2003) Oxygen and iron regulation of iron regulatory protein 2. J Biol Chem 278(41): 40337-40342.

Harrington K A, Augood S J, Kingsbury A E, Foster O J and Emson P C (1996) Dopamine transporter (Dat) and synaptic vesicle amine transporter (VMAT2) gene expression in the substantia nigra of control and Parkinson's disease. Brain Res Mol Brain Res 36(1): 157-162.

Hashimoto M, Bar-On P, Ho G, Takenouchi T, Rockenstein E, Crews L and Masliah E (2004) Betasynuclein regulates Akt activity in neuronal cells. A possible mechanism for neuroprotection in Parkinson's disease. J Biol Chem 279(22): 23622-23629.

Hassouna I, Wickert H, Zimmermann M, Gillardon F. (1996) Increase in bax expression in substantia nigra following 1-methyl-4-phenyl-1,2,3,6-tetrahydropyridine (MPTP) treatment of mice. Neurosci Lett. 204(1-2):85-8.

Hay J C, Hirling H and Scheller R H (1996) Mammalian vesicle trafficking proteins of the endoplasmic reticulum and Golgi apparatus. J Biol Chem 271(10): 5671-5679.

Hjelle J J and Petersen D R (1983) Hepatic aldehyde dehydrogenases and lipid peroxidation. Pharmacol Biochem Behav 18 Suppl 1:155-160.

Hoehn M M and Yahr M D (1967) Parkinsonism: onset, progression and mortality. Neurology 17(5): 427-442.

Hosack D, Dennis G, Sherman B, Lane H and Lempicki R (2003) Identifying biological themes within lists of genes with EASE. Genome Biol 4(10): R70.

Iacopino A M, Christakos S. (1990) Specific reduction of calcium-binding protein (28-kilodalton calbindin-D) gene expression in aging and neurodegenerative diseases. Proc Natl Acad Sci U S A. 87(11):4078-82.

Imai Y, Soda M, Hatakeyama S, Akagi T, Hashikawa T, Nakayama K I and Takahashi R (2002) CHIP is associated with Parkin, a gene responsible for familial Parkinson's disease, and enhances its ubiquitin ligase activity. Mol Cell 10(1): 55-67.

Jankovic J, Rajput A H, McDermott M P, Perl D P. (2000) The evolution of diagnosis in early Parkinson disease. Parkinson Study Group. Arch Neurol. 57(3):369-72.

Jayanthi S, Deng X, Bordelon M, McCoy M T, Cadet J L. (2001) Methamphetamine causes differential regulation of pro-death and anti-death Bcl-2 genes in the mouse neocortex. FASEB J. 15(10):1745-52.

Jellinger K A (2003) Neuropathological spectrum of synucleinopathies. Mov Disord 18 (Suppl 6):S2-12.

Jenner P (1998) Oxidative mechanisms in nigral cell death in Parkinson's disease. Mov Disord (Suppl 13): 24-34.

Jenner P and Olanow C W (1996) Oxidative stress and the pathogenesis of Parkinson's disease. Neurology 47(6 Suppl 3): S161-170.

Kamura T, Koepp D M, Conrad M N, Skowyra D, Moreland R J, Iliopoulos O, Lane W S, Kaelin W G, Jr., Elledge S J, Conaway R C, Harper J W and Conaway J W (1999) Rbx1, a component of the VHL tumor suppressor complex and SCF ubiquitin ligase. Science 284(5414): 657-661.

Kitada T, Asakawa S, Hattori N, Matsumine H, Yamamura Y, Minoshima S, Yokochi M, Mizuno Y, Shimizu N. (1998) Mutations in the parkin gene cause autosomal recessive juvenile parkinsonism. Nature. 392(6676):605-8.

Klucken J, Shin Y, Masliah E, Hyman B T and McLean P J (2004) Hsp70 Reduces alpha-Synuclein Aggregation and Toxicity. J Biol Chem 279(24): 25497-25502.

Koller W C. (2002) Treatment of early Parkinson's disease. Neurology. 2002 February 26; 58(4 Suppl 1):S79-86.

Laprade N, Soghomonian J J. (1999) Gene expression of the GAD67 and GAD65 isoforms of glutamate decarboxylase is differentially altered in subpopulations of striatal neurons in adult rats lesioned with 6-OHDA as neonates. Synapse. 33(1):36-48.

LaVaute T, Smith S. Cooperman S, Iwai K, Land W, Meyron-Holtz E, Drake S K, Miller G, Abu-Asab M, Tsokos M, Switzer R, 3rd, Grinberg A, Love P, Tresser N and Rouault T A (2001) Targeted deletion of the gene encoding iron regulatory protein-2 causes misregulation of iron metabolism and neurodegenerative disease in mice. Nat Genet 27(2): 209-214.

Le H N, Frim D M. (2002) Gene therapy for Parkinson's disease. Expert Opin Biol Ther. 2(2):151-61.

Lee J W, Bae S H, Jeong J, Kim S H and Kim K W (2004) Hypoxia-inducible factor (HIF-1)alpha: its protein stability and biological functions. Exp Mol Med 29; 36(1): 1-12.

Liani E, Eyal A, Avraham E, Shemer R, Szargel R, Berg D, Bomemann A, Riess O, Ross C A, Rott R and Engelender S (2004) Ubiquitylation of synphilin-1 and alpha-synuclein by SIAH and its presence in cellular inclusions and Lewy bodies imply a role in Parkinson's disease. Proc Natl Acad Sci USA 101(15): 5500-5505.

Lukiw W J and Bazan N G (1997) Cyclooxygenase 2 RNA message abundance, stability, and hypervariability in sporadic Alzheimer neocortex. J Neurosci Res 50(6): 937-945.

Luo J, Kaplitt M G, Fitzsimons H L, Zuzga D S, Liu Y, Oshinsky M L, During M J. (2002) Subthalamic GAD gene therapy in a Parkinson's disease rat model. 298(5592):425-9.

Mardh G and Vallee B L (1986) Human class I alcohol dehydrogenases catalyze the interconversion of alcohols and aldehydes in the metabolism of dopamine. Biochemistry 25(23): 7279-7282.

McNaught K S, Belizaire R, Jenner P, Olanow C W and Isacson O (2002) Selective loss of 20S proteasome alpha-subunits in the substantia nigra pars compacta in Parkinson's disease. Neurosci Lett 326(3): 155-158.

McNaught K S, Belizaire R, Isacson O, Jenner P and Olanow C W (2003) Altered proteasomal function in sporadic Parkinson's disease. Exp Neurol 179(1): 38-46.

McBride J L, Kordower J H. (2002) Neuroprotection for Parkinson's disease using viral vector-mediated delivery of GDNF. Prog Brain Res. 2002; 138:421-32.

Meredith G E, Halliday G M and Totterdell S (2004) A critical review of the development and importance of proteinaceous aggregates in animal models of Parkinson's disease: new insights into Lewy body formation. Parkinsonism Relat Disord 10(4): 191-202.

Meyron-Holtz E G, Ghosh M C, Iwai K, LaVaute T, Brazzolotto X, Berger U V, Land W, Ollivierre-Wilson H, Grinberg A, Love P and Rouault T A (2004) Genetic ablations of iron regulatory proteins 1 and 2 reveal why iron regulatory protein 2 dominates iron homeostasis. EMBO J 23(2): 386-395.

Minchenko O, Opentanova I and Caro J (2003) Hypoxic regulation of the 6-phosphofructo-2-kinase/fructose-2,6-bisphosphatase gene family (PFKFB-1-4) expression in vivo. FEBS Lett 554(3): 264-270.

Monville C. (2002) Gene therapy in Parkinson's disease: dream or reality? Neuroreport. 13(6):743.

Murata S, Chiba T and Tanaka K (2003) CHIP: a quality-control E3 ligase collaborating with molecular chaperones. Int J Biochem Cell Biol 35(5): 572-578.

Nagai Y, Ueno S, Saeki Y, Soga F, Hirano M, Yanagihara T. (1996) Decrease of the D3 dopamine receptor mRNA expression in lymphocytes from patients with Parkinson's disease. Neurology. 46(3):791-5.

Olanow C W and Youdim M B (1996) Iron and neurodegeneration: prospects for neuroprotection, In: Olanow C W, Jenner P and Youdim M B (eds) Neurodegeneration and Neuroprotection in Parkinson's Disease, Academic Press Edition, London, pp 55-69.

Onlanow, C., Jenner, and Brooks, D., Dopamine agonists and neuroprotection in Parkinson's disease. In C. Onlanow and P. Jenner (Eds.), Beyond the decade of the Brain. Neuroprotection in Parkinson's disease, Vol. 3, Wells Medical Limited, Kentum, UK, 1998, pp. 331-340.

Olanow C W. (2002) Surgical therapy for Parkinson's disease. Eur J Neurol. 9 Suppl 3:31-9.

Ostrerova-Golts N, Petrucelli L, Hardy J, Lee J M, Farer M and Wolozin B (2000) The A53T alphasynuclein mutation increases iron-dependent aggregation and toxicity. J Neurosci 20(16): 6048-6054.

Ouimet C C, Hemmings H C, Jr. and Greengard P (1989) ARPP-21, a cyclic AMP-regulated phosphoprotein enriched in dopamine-innervated brain regions. II. Immunocytochemical localization in rat brain. J Neurosci 9(3): 865-875.

Padmanabhan J, Park D S, Greene L A and Shelanski M L (1999) Role of cell cycle regulatory proteins in cerebellar granule neuron apoptosis. J Neurosci 19(20): 8747-8756.

Polymeropoulos M H, Lavedan C, Leroy E, Ide S E, Dehejia A, Dutra A, Pike B, Root H, Rubenstein J, Boyer R, Stenroos E S, Chandrasekharappa S, Athanassiadou A, Papapetropoulos T, Johnson W G, Lazzarini A M, Duvoisin R C, Di Iorio G, Golbe L I, Nussbaum R L. (1997) Mutation in the alpha-synuclein gene identified in families with Parkinson's disease. Science. 276(5321):2045-7.

Ponka P (2004) Hereditary causes of disturbed iron homeostasis in the central nervous system. Ann N Y Acad Sci 1012:267-281.

Ribeiro C S, Carneiro K, Ross C A, Menezes J R and Engelender S (2002) Synphilin-1 is developmentally localized to synaptic terminals, and its association with synaptic vesicles is modulated by alphasynuclein. J Biol Chem 277(26): 23927-23933.

Riederer P, Sofic E, Rausch W D, Schmidt B, Reynolds G P, Jellinger K and Youdim M B H (1989) Transition metals, ferritin, glutathione, and ascorbic acid in parkinsonian brains. J Neurochem 52(2): 515-520.

Riederer P, Sian J, Gerlach M. (2000) Is there neuroprotection in Parkinson syndrome? J Neurol. 247 Suppl 4:IV/8-11.

Riederer P, Gille G, Muller T, Przuntek H, Reichmann H, Riess O, Schwartz A, Schwarz J, Vogt T. (2002) Practical importance of neuroprotection in Parkinson's disease. J Neurol. 2002 October; 249 Suppl 3:111/53-6.

Schulz H, Weber B and Wiedmann C (2004) Quantitative real-time PCR-eine frage der normalisierung. BIO Spektrum 1:99-101.

Semple C A (2003) The comparative proteomics of ubiquitination in mouse. GenomeRes 13(6B): 1389-1394.

Sherman M Y and Goldberg A L (2001) Cellular defenses against unfolded proteins: a cell biologist thinks about neurodegenerative diseases. Neuron 29(1): 15-32.

Snyder H, Mensah K, Theisler C, Lee J, Matouschek A and Wolozin B (2003) Aggregated and monomeric alpha-synuclein bind to the S6′ proteasomal protein and inhibit proteasomal function. J Biol Chem 278(14): 11753-11759.

Soghomonian J J, Laprade N. (1997) Glutamate decarboxylase (GAD67 and GAD65) gene expression is increased in a subpopulation of neurons in the putamen of Parkinsonian monkeys. Synapse. 27(2):122-32.

Senior K. (2002) Gene therapy for Parkinson's disease. Drug Discov Today. 7(2):88-9.

Soto-Otero R, Mendez-Alvarez E, Hermida-Ameijeiras A, Lopez-Real A M, Labandeira-Garcia J L. (2002) Effects of (−)-nicotine and (−)-cotinine on 6-hydroxydopamine-induced oxidative stress and neurotoxicity: relevance for Parkinson's disease. Biochem Pharmacol 64(1): 125-35.

Staropoli J F, McDermott C, Martinat C, Schulman B, Demireva E and Abeliovich A (2003) Parkin is a component of an SCF-like ubiquitin ligase complex and protects postmitotic neurons from kainate excitotoxicity. Neuron 37(5): 735-749.

Stenoien D L, Cummings C J, Adams H P, Mancini M G, Patel K, DeMartino G N, Marcelli M, Weigel N L and Mancini M A (1999) Polyglutamine-expanded androgen receptors form aggregates that sequester heat shock proteins, proteasome components and SRC-1, and are suppressed by the HDJ-2 chaperone. Hum Mol Genet 8(5): 731-741.

Strickland E, Hakala K, Thomas P J and DeMartino G N (2000) Recognition of Misfolding Proteins by PA700, the Regulatory Subcomplex of the 26 S Proteasome. J Biol Chem 275(8): 5565-5572.

Svaren J, Ehrig T, Abdulkadir S A, Ehrengruber M U, Watson M A and Milbrandt J (2000) EGR1 Target Genes in Prostate Carcinoma Cells Identified by Microarray Analysis. J Biol Chem 275(49): 38524-38531.

Tanaka M, Kim Y M, Lee G, Junn E, Iwatsubo T and Mouradian M M (2004) Aggresomes formed by alpha-synuclein and synphilin-1 are cytoprotective. J Biol Chem 279(6): 4625-4631.

Tenenbaum L, Chtarto A, Lehtonen E, Blum D, Baekelandt V. Velu T, Brotchi J, Levivier M. (2002) Neuroprotective gene therapy for Parkinson's disease. Curr Gene Ther. 2(4):451-83.

Tsou K, Girault J A and Greengard P (1993) Dopamine D1 agonist SKF 38393 increases the state of phosphorylation of ARPP-21 in substantia nigra. J Neurochem 60(3): 1043-1046.

Tsukahara F, Yoshioka T and Muraki T (2000) Molecular and functional characterization of HSC54, a novel variant of human heat-shock cognate protein 70. Mol Pharmacol 58(6): 1257-1263.

Turnbull S, Tabner B J, El-Agnaf O M, Moore S, Davies Y and Allsop D (2001) alpha-Synuclein implicated in Parkinson's disease catalyses the formation of hydrogen peroxide in vitro. Free Radic Biol Med 30(10): 1163-1170.

Ugozzoli L A, Chinn D and Hamby K (2002) Fluorescent multicolor multiplex homogeneous assay for the simultaneous analysis of the two most common hemochromatosis mutations. Anal Biochem 307(1): 47-53.

Vandesompele J, De Preter K, Pattyn F, Poppe B, Van Roy N, De Paepe A and Speleman F (2002) Accurate normalization of real-time quantitative RT-PCR data by geometric averaging of multiple internal control genes. Genome Biol 3(7): RESEARCH0034.

Verdaguer E, Garcia-Jorda E, Canudas A M, Dominguez E, Jimenez A, Pubill D, Escubedo E, Pallas J C and Camins A (2002) Kainic acid-induced apoptosis in cerebellar granule neurons: an attempt at cell cycle re-entry. Neuroreport 13(4): 413-416.

Voges D, Zwickl P and Baumeister W (1999) The 26S proteasome: a molecular machine designed for controlled proteolysis. Annu Rev Biochem 68:1015-1068.

Wakabayashi K, Engelender S, Yoshimoto M, Tsuji S, Ross C A and Takahashi H (2000) Synphilin-1 is present in Lewy bodies in Parkinson's disease. Ann Neurol 47(4): 521-523.

Wang H Y, Lin W, Dyck J A, Yeakley J M, Songyang Z, Cantley L C and Fu X D (1998) SRPK2: a differentially expressed SR protein-specific kinase involved in mediating the interaction and localization of pre-mRNA splicing factors in mammalian cells. J Cell Biol 140(4): 737-750.

Wang J, Chen G, Muckenthaler M, Galy B, Hentze M W and Pantopoulos K (2004) Ironmediated degradation of IRP2, an unexpected pathway involving a 2-oxoglutarate-dependent oxygenase activity. Mol Cell Biol 24(3): 954-965.

Wendler F and Tooze S (2001) Syntaxin 6: the promiscuous behaviour of a SNARE protein. Traffic 2(9): 606-611.

Wu Y R, Wang C K, Chen C M, Hsu Y, Lin S J, Lin Y Y, Fung H C, Chang K H and Lee-Chen G J (2004) Analysis of heat-shock protein 70 gene polymorphisms and the risk of Parkinson's disease. Hum Genet 114(3): 236-241.

Yamaguchi T, Kuraishi Y, Minami M, Nakai S, Hirai Y, Satoh M. (1991) Methamphetamine-induced expression of interleukin-1 beta mRNA in the rat hypothalamus. Neurosci Lett. 128(1):90-2.

Youdim M B, Ben-Shachar D and Riederer P (1993) The possible role of iron in the etiopathology of Parkinson's disease. Mov Disord 8(1): 1-12.

Youdim M B H and Riederer P (1997) Understanding Parkinson's disease. Sci Am 276(1): 52-59.

Youdim M B H and Riederer P (2004) Iron in the brain, normal and pathological, In: Adelman G and Smith B (eds) Encyclopedia of Neuroscience.

Zhang J and Goodlett D R (2004) Proteomic approach to studying Parkinson's disease. Mol Neurobiol 29(3): 271-288.

Zigmond M J, Berger T W, Grace A A, Stricker E M. (1989) Compensatory responses to nigrostriatal bundle injury. Studies with 6-hydroxydopamine in an animal model of parkinsonism. Mol Chem Neuropathol. 10(3): 185-200.

Zinsmaier K E and Bronk P (2001) Molecular chaperones and the regulation of neurotransmitter exocytosis. Biochem Pharmacol 62(1): 1-11.

1-15. (canceled)

16. A method for diagnosis, prognosis and/or follow up of Parkinson's disease comprising the use of molecular markers, wherein said molecular markers are one or more genes with altered expression pattern, or gene products thereof, said genes being selected from the group consisting of: ALDH1A1, ARPP-21, HSPA8, SKP1A, SLC18A2, SRPK2, TMEFF1, TRIM36, ADH5, PSMA3, PSMA2, PSMA5, PSMC4, HIP2, PACE4, COX6A1, PFKP, OXCT, GBE1, UQCRC2, LANCL1, TRIP15, PIK3CA, PLCL1, GNG5, GNAI1, VEGF, RHOB, NR4A2, SCL31A2, SCP2, PIGH, ARIH2, GMPR2, PP, IKBKAP, PRKACB, PTPRN2, BCAS2, IARS, PPP1R8, SEP15, TAF9, ZFP103, WRB, TMEM4, SMARCA3, FMR1, PDE6D, SGCE, AUH, SLC16A7, ATP6V1E1, UGTREL1, SEC22L1, CD9, CDH19, DUSP1, HSA6591, ACTR3, KIF2, TUBB2, ASPA, HELO1, C3orf4, CBR1, XPOT, LOC51142, NY-REN-45, SET0-2, EGLN1, EIF4EBP2, LGALS9, LOC56920, LRP6, MAN2B1, PARVA, PENK, SELPLG, SPHK1, SRRM2, ZSIG11, ITGB3BP, ITGAM, COL18A1, TM4SF9, LAMB2, HS3ST2, TSTA3, COL5A3, PALM, MYOM1, FLNB, HMBS, KRT2A, CSK, NUDC, HYPE, GAK, SIAT1, CSF1R, ICSBP1, CD22, ERCC1, DNAJB5, TRAF3, MMP9, EIF4G1, RPL36, SRPK1, CSNK1G2, RPS6KA1, JIK, LNK, INPP5D, TCOF1, NAPG, SLC19A1, ITSN1, LOC51035, PMVK, C21orf2, EFEMP2, TBL1X, APRT, SPUF, GLTSCR2, ADIR, PSCD4, CBFA2T1, CUGBP1, ING4, STAT6, ZNF239, TAL1, TAF11, MXD4, RDHL, LOC51157, LRP6, MBD3, and C9orf7.

17. A method according to claim 16, wherein said molecular markers are one or more genes selected from the group consisting of: ALDH1A1, ARPP-21, HSPA8, SKP1A, SLC18A2, SRPK2, TMEFF1, TRIM36, ADH5, PSMA3, PSMA2, PSMA5, PSMC4, EGLN1, EIF4EBP2, LGALS9, LOC56920, LRP6, MAN2B1, PARVA, PENK, SELPLG, SPHK1, SRRM2, and ZSIG11.

18. A method according to claim 16, which comprises detecting a decreased level of expression of one or more genes selected from the group consisting of: ALDH1A1, ARPP-21, HSPA8, SKP1A, SLC18A2, SRPK2, TMEFF1, TRIM36, ADH5, PSMA3, PSMA2, PSMA5, PSMC4, HIP2, PACE4, COX6A1, PFKP, OXCT, GBE1, UQCRC2, LANCL1, TRIP15, PIK3CA, PLCL1, GNG5, GNAI1, VEGF, RHOB, NR4A2, SCL31A2, SCP2, PIGH, ARIH2, GMPR2, PP, IKBKAP, PRKACB, PTPRN2, BCAS2, IARS, PPP1R8, SEP15, TAF9, ZFP103, WRB, TMEM4, SMARCA3, FMR1, PDE6D, SGCE, AUH, SLC16A7, ATP6V1E1, UGTREL1, SEC22L1, CD9, CDH19, DUSP1, HSA6591, ACTR3, KIF2, TUBB2, ASPA, HELO1, C3orf4, CBR1, XPOT, LOC51142, NY-REN-45, SET0-2, and/or an increased level of expression of one or more genes selected from the group consisting of: EGLN1, EIF4EBP2, LGALS9, LOC56920, LRP6, MAN2B1, PARVA, PENK, SELPLG, SPHK1, SRRM2, ZSIG11, ITGB3BP, ITGAM, COL18A1, TM4SF9, LAMB2, HS3ST2, TSTA3, COL5A3, PALM, MYOM1, FLNB, HMBS, KRT2A, CSK, NUDC, HYPE, GAK, SIAT1, CSF1R, ICSBP1, CD22, ERCC1, DNAJB5, TRAF3, MMP9, EIF4G1, RPL36, SRPK1, CSNK1G2, RPS6KA1, JIK, LNK, INPP5D, TCOF1, NAPG, SLC19A1, ITSN1, LOC51035, PMVK, C21orf2, EFEMP2, TBL1X, APRT, SPUF, GLTSCR2, ADIR, PSCD4, CBFA2T1, CUGBP1, ING4, STAT6, ZNF239, TAL1, TAF11, MXD4, RDHL, LOC51157, LRP6, MBD3, and C9orf7.

19. A method according to claim 18, which comprises detecting a decreased level of expression of one or more genes selected from the group consisting of: ALDH1A1, ARPP-21, HSPA8, SKP1A, SLC18A2, SRPK2, TMEFF1, TRIM36, ADH5, PSMA3, PSMA2, PSMA5, PSMC4, and/or an increased level of expression of one or more genes selected from the group consisting of: EGLN1, EIF4EBP2, LGALS9, LOC56920, LRP6, MAN2B1, PARVA, PENK, SELPLG, SPHK1, SRRM2, ZSIG11.

20. A method for diagnosing occurrence of Parkinson's disease in an individual exhibiting Parkinsonian-like symptoms, comprising detecting in a sample obtained from said individual a decreased level of expression of one or more genes selected from the group consisting of: ALDH1A1, ARPP-21, HSPA8, SKP1A, SLC18A2, SRPK2, TMEFF1, TRIM36, ADH5, PSMA3, PSMA2, PSMA5, PSMC4, HIP2, PACE4, COX6A1, PFKP, OXCT, GBE1, UQCRC2, LANCL1, TRIP15, PIK3CA, PLCL1, GNG5, GNAI1, VEGF, RHOB, NR4A2, SCL31A2, SCP2, PIGH, ARIH2, GMPR2, PP, IKBKAP, PRKACB, PTPRN2, BCAS2, IARS, PPP1R8, SEP15, TAF9, ZFP103, WRB, TMEM4, SMARCA3, FMR1, PDE6D, SGCE, AUH, SLC16A7, ATP6V1E1, UGTREL1, SEC22L1, CD9, CDH19, DUSP1, HSA6591, ACTR3, KIF2, TUBB2, ASPA, HELO1, C3orf4, CBR1, XPOT, LOC51142, NY-REN-45, and SET0-2, and/or an increased level of expression of one or more genes selected from the group consisting of: EGLN1, EIF4EBP2, LGALS9, LOC56920, LRP6, MAN2B1, PARVA, PENK, SELPLG, SPHK1, SRRM2, ZSIG11, ITGB3BP, ITGAM, COL18A1, TM4SF9, LAMB2, HS3ST2, TSTA3, COL5A3, PALM, MYOM1, FLNB, HMBS, KRT2A, CSK, NUDC, HYPE, GAK, SIAT1, CSF1R, ICSBP1, CD22, ERCC1, DNAJB5, TRAF3, MMP9, EIF4G1, RPL36, SRPK1, CSNK1G2, RPS6KA1, JIK, LNK, INPP5D, TCOF1, NAPG, SLC19A1, ITSN1, LOC51035, PMVK, C21orf2, EFEMP2, TBL1X, APRT, SPUF, GLTSCR2, ADIR, PSCD4, CBFA2T1, CUGBP1, ING4, STAT6, ZNF239, TAL1, TAF11, MXD4, RDHL, LOC51157, LRP6, MBD3, and C9orf7, wherein said decreased and/or increased level of expression of said genes is diagnostic of Parkinson's disease.

21. A method according to claim 20 comprising detecting a decreased level of expression of one or more genes selected from the group consisting of: ALDH1A1, ARPP-21, HSPA8, SKP1A, SLC18A2, SRPK2, TMEFF1, TRIM36, ADH5, PSMA3, PSMA2, PSMA5, and PSMC4, and/or an increased level of expression of one or more genes selected from the group consisting of: EGLN1, EIF4EBP2, LGALS9, LOC56920, LRP6, MAN2B1, PARVA, PENK, SELPLG, SPHK1, SRRM2, and ZSIG11.

22. A method according to claim 20, wherein said sample is blood, serum or biopsy of skin from the tested individual.

23. The method according to claim 20, which comprises detecting a gene product expressed by said gene and the gene product is a protein expressed by the gene or RNA transcribed from the gene, or both.

24. The method according to claim 23, wherein said detection comprises assaying for the presence of a protein expressed by the gene by contacting the sample with an antibody, or a fragment thereof, that binds to the protein.

25. The method according to claim 23, which comprises detection in said sample of decreased or increased levels of RNA transcripts.

26. The method according to claim 25, wherein detection of the RNA transcripts is carried out by a method selected from the group consisting of Northern blots, RNase protection assays (RPA), nucleic acid probe arrays, real-time quantitative reverse-transcription PCR (RT-PCR), dot blot assays and in-situ hybridization.

27. A method for screening for an agent useful for treating Parkinson's disease, which comprises identifying an agent that upregulates the expression of one or more genes selected from the group consisting of ALDH1A1, ARPP-21, HSPA8, SKP1A, SLC18A2, SRPK2, TMEFF1, TRIM36, ADH5, PSMA3, PSMA2, PSMA5, PSMC4, HIP2, PACE4, COX6A1, PFKP, OXCT, GBE1, UQCRC2, LANCL1, TRIP15, PIK3CA, PLCL1, GNG5, GNAI1, VEGF, RHOB, NR4A2, SCL31A2, SCP2, PIGH, ARIH2, GMPR2, PP, IKBKAP, PRKACB, PTPRN2, BCAS2, IARS, PPP1R8, SEP15, TAF9, ZFP103, WRB, TMEM4, SMARCA3, FMR1, PDE6D, SGCE, AUH, SLC16A7, ATP6V1E1, UGTREL1, SEC22L1, CD9, CDH19, DUSP1, HSA6591, ACTR3, KIF2, TUBB2, ASPA, HELO1, C3orf4, CBR1, XPOT, LOC51142, NY-REN-45, and SETO-2, or down-regulates the expression of one or more genes selected from the group consisting of: EGLN1, EIF4EBP2, LGALS9, LOC56920, LRP6, MAN2B1, PARVA, PENK, SELPLG, SPKH1, SRRM2, ZSIG11, ITGB3BP, ITGAM, COL18A1, TM4SF9, LAMB2, HS3ST2, TSTA3, COL5A3, PALM, MYOM1, FLNB, HMBS, KRT2A, CSK, NUDC, HYPE, GAK, SIAT1, CSF1R, ICSBP1, CD22, ERCC1, DNAJB5, TRAF3, MMP9, EIF4G1, RPL36, SRPK1, CSNK1G2, RPS6KA1, JIK, LNK, INPP5D, TCOF1, NAPG, SLC19A1, ITSN1, LOC51035, PMVK, C21orf2, EFEMP2, TBL1X, APRT, SPUF, GLTSCR2, ADIR, PSCD4, CBFA2T1, CUGBP1, ING4, STAT6, ZNF239, TAL1, TAF11, MXD4, RDHL, LOC51157, LRP6, MBD3, and C9orf7.

28. A method according to claim 27, which comprises identifying an agent that upregulates the expression of one or more genes selected from the group consisting of: ALDH1A1, ARPP-21, HSPA8, SKP1A, SLC18A2, SRPK2, TMEFF1, TRIM36, ADH5, PSMA3, PSMA2, PSMA5, PSMC4, or down-regulates the expression of one or more genes selected from the group consisting of: EGLN1, EIF4EBP2, LGALS9, LOC56920, LRP6, MAN2B1, PARVA, PENK, SELPLG, SPHK1, SRRM2, ZSIG11.
